(19) United States
(12) Patent Application Publication
(10) Pub. No.: US 2015/0328099 A1
(43) Pub. Date: Nov. 19, 2015

(54) Antitranspirant Deodorant Cosmetic Composition Having Dermo-Calming Action

(71) Applicant: NATURA COSMÉTICOS S.A, Itapecerica da Serra (BR)

(72) Inventors: Silvania ANGELINO DOS SANTOS TEODORO, Itapecerica da Serra (BR); Selma NASCIMENTO, Itapecerica da Serra (BR); Elisangela COSTA GAMA, Itapecerica da Serra (BR); Joice SAVIETTO, Itapecerica da Serra (BR)

(73) Assignee: NATURA COSMÉTICOS S.A, Itapecerica da Serra (BR)

(21) Appl. No.: 14/279,773

(22) Filed: May 16, 2014

(51) Int. Cl. A61K 8/34; A61Q 15/00; A61K 8/86; A61K 8/42; A61K 8/58; A61K 8/26; A61Q 19/00

(52) U.S. Cl. CPC A61K 8/34; A61K 8/26; A61Q 15/00; A61Q 19/005; A61K 8/42; A61K 8/585; A61K 8/86; A61K 2800/75; A61K 2800/592

(57) ABSTRACT

The present invention relates to antiperspirant deodorant cosmetic compositions comprising 2-methyl-5-cyclohexylpentanol, aluminum hydrochloride, hydroxypropylic starch phosphate, pantenol, and cosmetically acceptable adjuvants. The compositions exhibit deodorant and antiperspirant actions, sensorial emollient characteristics, and a protective film, and further provide a dermo-calming action on the skin for after-depilation use.

### FIELD OF THE INVENTION

The present invention relates to antiperspirant deodorant cosmetic compositions with dermo-calming action for after-depilation sensitized skin, applicable in the cosmetic, hygiene and personal-care industry.

### BACKGROUND OF THE INVENTION

Deodorants are intended for perfuming the body and, in general, they contain antimicrobial components, particularly antibacterial and antifungal, which eliminate bacteria and fungi that cause bad smell on the skin. Deodorants may be applied to armpits to perfume them and diminish the odors generated in this body region; however they do not prevent perspiration.

Antiperspirants have the function of controlling the perspiration by inhibiting or reducing it, thus guaranteeing protection against sweat, besides having antimicrobial action, eliminating the microorganisms that cause bad smell. The antiperspirant action is due to the fact that this product acts by forming a blocking film that prevent sweat from coming out, without causing damage to one's health.

It is known that depilation, be it by wax, laser or nippers, induces cutaneous irritation in determined persons and body regions.

Among the measures which one should take with depilation are: non-exposure to direct sunshine, skin cleaning, use of light and loose clothes, as well as attention to deodorants, creams and body oils used after the depilatory procedure. For this reason, care with the skin has become essential, although cutaneous irritations still occur, even with such care.

In this regard, the search for specific products for this purpose, particularly for deodorants that provide relief of irritation caused on the skin, i.e., sensitized by depilation, becomes critical. Also, in addition to deodorants that relieve irritation, it becomes essential to develop products that group deodorant characteristics to fight odor, antiperspirant for reducing sweat, and still that have a dermo-calming action on the skin.

A few examples of prior-art documents related to the area of the present invention, particularly antiperspirant deodorants, are presented hereinafter.

Patent application PI0924661-4, published on Nov. 21, 2012, in the name of Symrise AG, relates to ω-cycle-hexylalkan-1-ols, to the use of said compounds as antimicrobial agents for the treatment of odor on the body or for the preparation of an antimicrobial cosmetic or pharmaceutical formulation. Said PI0924661-4 further describes antimicrobial formulations containing, for instance, 2-methyl-cyclohexylpentanol. However, said document does not mention or suggest the use of said active or formulations thereof as antiperspirant deodorants with dermo-calming action, especially for after-depilation sensitized skin.

U.S. Pat. No. 8,115,033, published on Feb. 14, 2012, in the name of Symrise Ag relates to the chemical compound 3-(4-hydroxy-3-methoxyphenyl)-1-(4-hydroxyphenyl)-1-propanone, which is different from the chemical compound of the present invention called SymDeo® B125: 2-methyl-5-cyclohexylpentanol. In the same way as the above-cited document, U.S. Pat. No. 8,115,033 does not mention or suggest the use of the active presently described and claimed or its formulations as antiperspirant deodorants with dermo-calming action, or use on after-depilation sensitized skin. In addition, even if this were the case, this is an active ingredient that is not foreseen in the composition of the present invention.

U.S. Pat. No. 6,172,016, published on Jan. 9, 2001, in the name of Bush Boakes Allen Inc., is directed to the use of pentane derivatives as cosmetic ingredients. The difference with respect to the chemical compounds SymDeo of the present invention lies in the carbon to which the methyl radical is attached, which is the third one in the American patent, namely: 3-methyl-5-cyclohexylpentanol. Again, this is a document that foresees a deodorant composition without the after-depilation calming benefit for sensitized skins.

Therefore, the need to develop antiperspirant deodorant formulations with dermo-calming action still remains, particularly for sensitized skins, which, in addition to the deodorant and antiperspirant properties, will exhibit emollient sensorial characteristics and a protective film, and provide dermo-calming action on the skin. Such need is fully met with the composition of the present invention.

### SUMMARY OF THE INVENTION

The present invention relates to antiperspirant deodorant cosmetic compositions with dermo-calming action, which comprise as active ingredients, 2-methyl-5-cyclohexylpentanol and aluminum hydrochloride, as well as commercially acceptable adjuvants, directed to application in the cosmetic, hygiene and body-care industry.

### BRIEF DESCRIPTION OF THE FIGURES

FIG. 1 refers to the result of treatment average as a function of the test for instrumental evaluation of the effect of the composition of the invention (called 13-39540-03) versus control composition on erythema induced by the tape-stripping technique.

FIG. 2 refers to the result of variation percentage in the average of the test for instrumental evaluation of the effect of the composition of the present invention (called 13-39540-03) versus control composition on erythema induced by the tape stripping technique.

FIG. 3 refers to the result of the average difference as a function of the time T0 of the test for instrumental evaluation of the effect of the composition of the invention (called 13-39540-03) versus control composition on erythema induced by the tape-stripping technique.

FIG. 4 refers to the result of treatment average as a function of the time of the test for instrumental evaluation of the effect of the composition of the present invention (called 13-39540-04) versus control composition on erythema induced by the tape-stripping technique.

FIG. 5 refers to the result of variation percentage in the average of the test for instrumental evaluation of the effect of the composition of the invention (called 13-39540-04) versus control composition on erythema induced by the tape-stripping technique.

FIG. 6 refers to the result of average difference as a function of the time T0 of the test for instrumental evaluation of the effect of the composition of the invention (called 13039540-04) versus control composition on erythema induced by the tape-stripping technique.

FIG. 7 refers to the result of treatment average as a function of the time of the test for instrumental evaluation of the effect of the composition of the invention (called 13-39540-01) versus control composition on erythema induced by the tape-stripping technique.

FIG. 8 refers to the result of variation percentage in the average of the test for instrumental evaluation of the effect of the composition of the invention (called 13-39540-01) versus control composition on erythema induced by the tape-stripping technique.

FIG. 9 refers to the result of average difference as a function of the time T0 of the test for instrumental evaluation of the effect of the composition of the invention (called 13-39540-01) versus control composition on erythema induced by the tape-stripping technique.

FIG. 10 refers to the result of treatment average as a function of the time of the test for instrumental evaluation of the effect of the composition of the invention (called 13-39540-02) versus control composition on erythema induced by the tape-stripping technique.

FIG. 11 refers to the result of variation percentage in the average of the test for instrumental evaluation of the effect of the composition of the invention (called 13-39540-02) versus control composition on erythema induced by the tape-stripping technique.

FIG. 12 refers to the result of average difference as a function of the time T0 of the test for instrumental evaluation of the effect of the composition of the invention (called 13-39540-02) versus control composition on erythema induced by the tape-stripping technique.

FIG. 13 refers to the average value of the erythema (E) for the product and control evaluated as a function of the time referring to the test for evaluation of the reduction of the erythema by using the composition of the invention (called NT1123-12-A).

FIG. 14 refers to the average value of the reduction the erythema (% RE) for the product and control evaluated as a function of the time referring to the test for evaluation of the reduction of erythema by using the composition of the invention (called NT1123-12-A).

FIG. 15 refers to the average value of the intensity of the erythema (+a*) for the product and control evaluated referring to the test for evaluation of the reduction of erythema by using the composition of the invention (called NT1123-12-A).

FIG. 16 refers to percentage of reduction of the intensity of the erythema (% RIE) as a function of the time referring to the test for evaluation of the reduction of erythema by using the composition of the invention (called NT1123-13-A).

FIG. 17 refers to the result of treatment average as a function of the test for instrumental evaluation of the effect of the composition of the invention (called 12-33171-07) versus control composition on erythema induced by the tape-stripping technique.

FIG. 18 refers to the result of variation percentage in the average of the test for instrumental evaluation of the effect of the composition of the invention (called 12-33171-7) versus control composition on erythema induced by the tape-stripping technique.

FIG. 19 refers to the result of average difference as a function of the time T0 of the test for instrumental evaluation of the effect of the composition of the invention (called 12-33171-07) versus control composition on erythema induced by the tape-stripping technique.

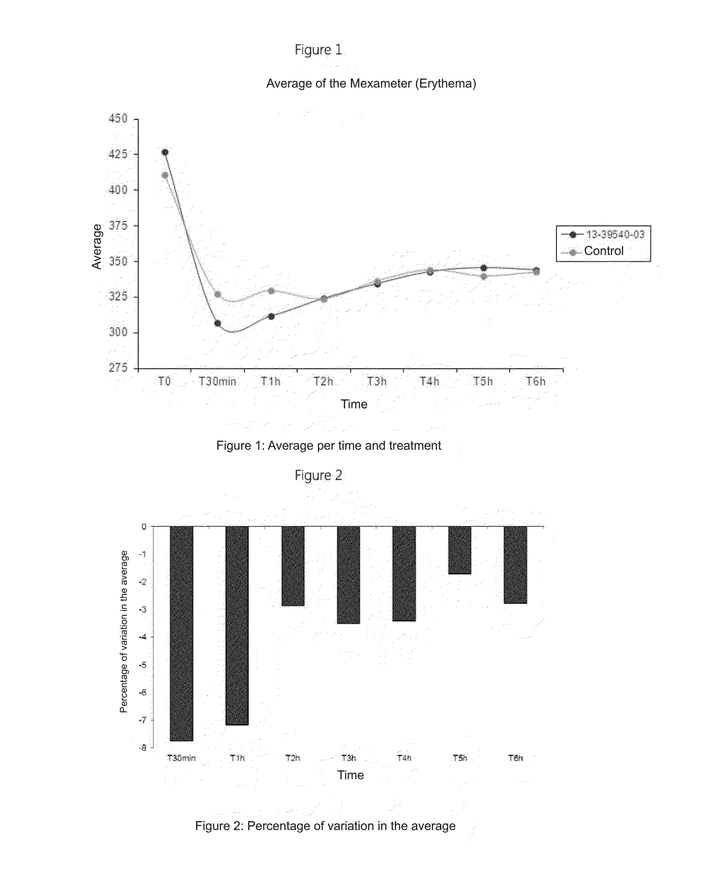

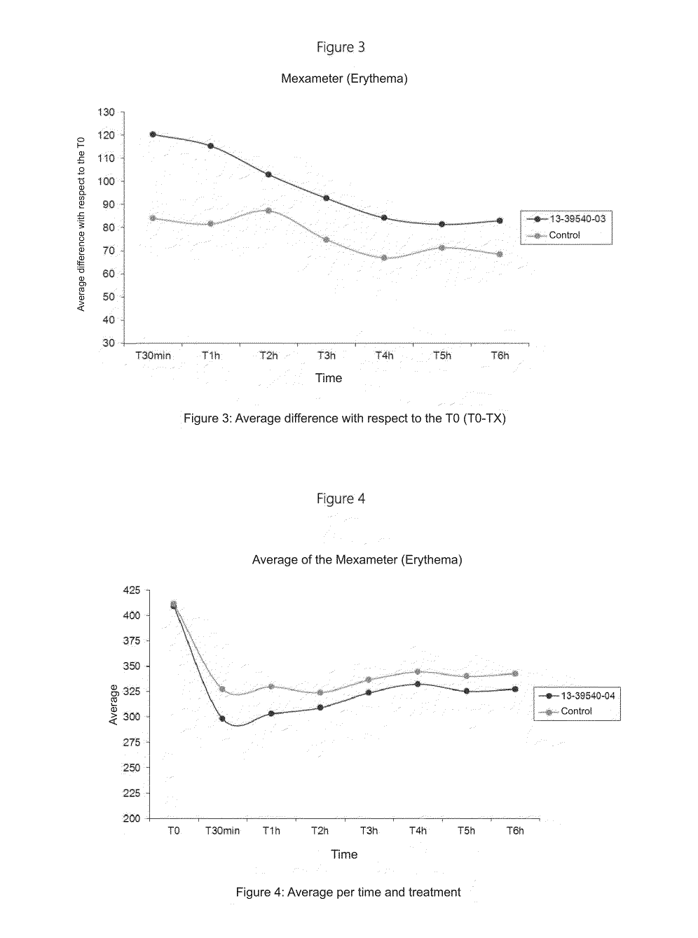

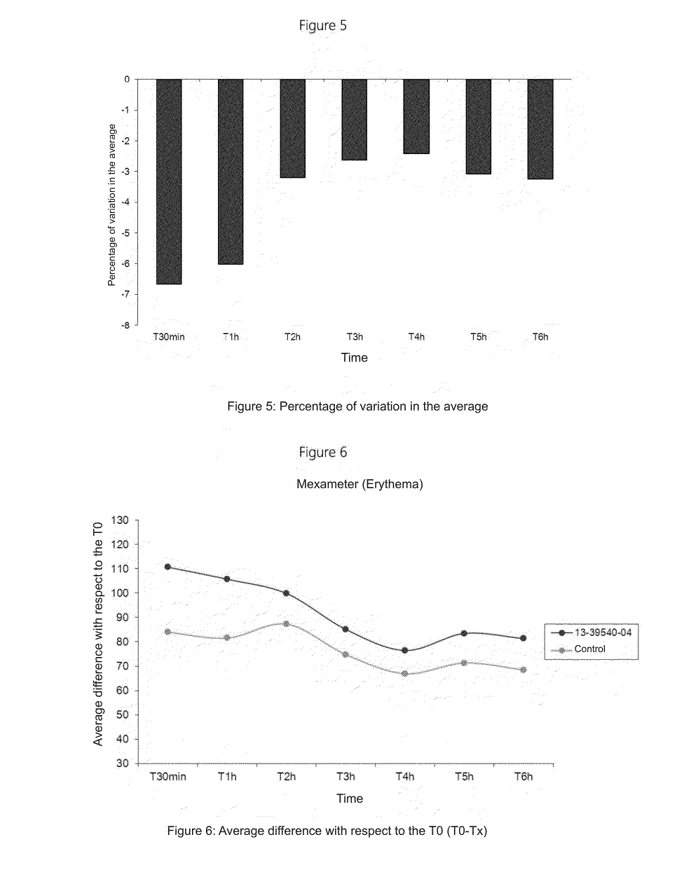

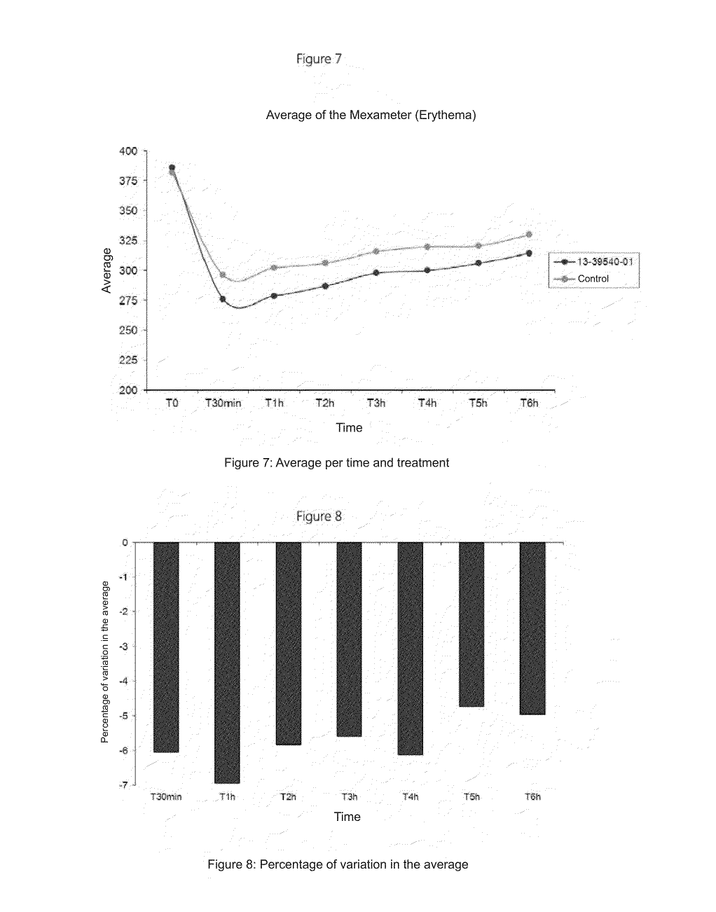

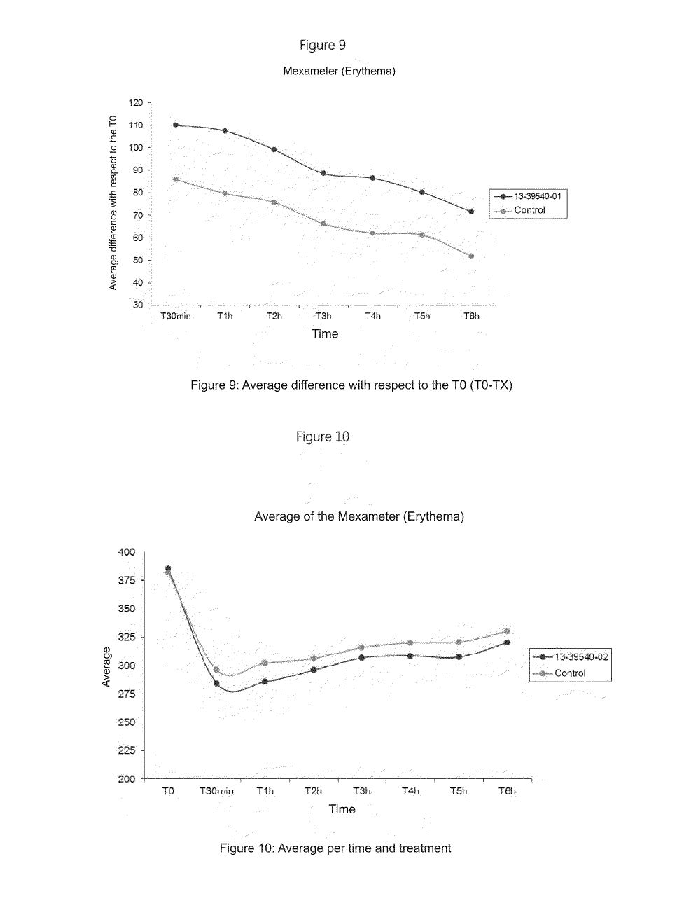

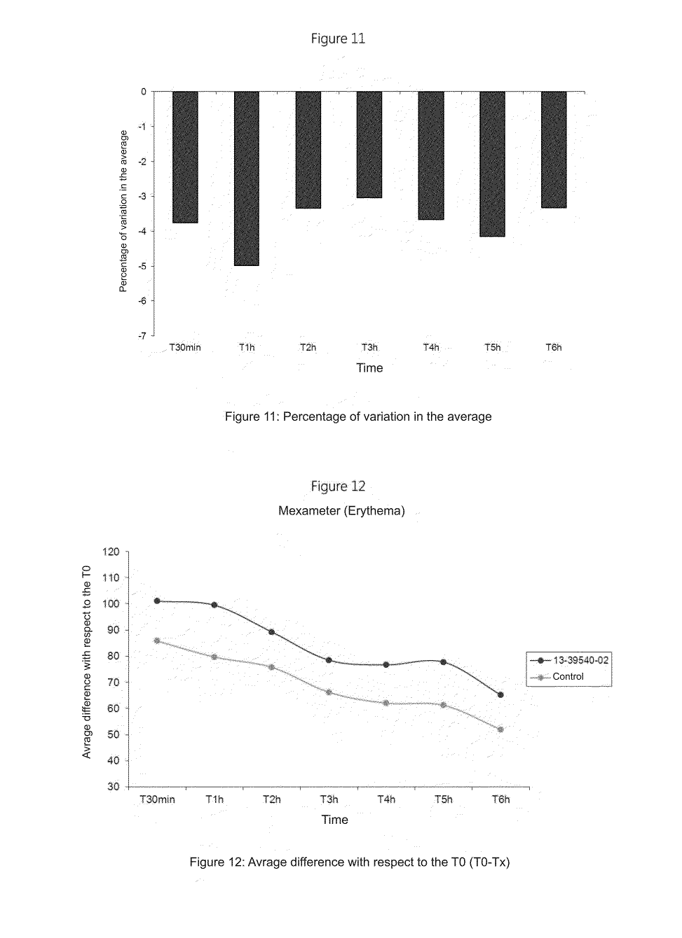

### DETAILED DESCRIPTION OF THE INVENTION

The antiperspirant deodorant cosmetic compositions with dermo-calming action of the present invention comprise 2-methyl-5-cyclohexylpentanol and aluminum hydrochloride and derivatives thereof as active ingredients, as well as cosmetically acceptably adjuvants.

Said adjuvants suitable for the purposes of the cosmetic compositions of the invention are selected, for example, from the group consisting of demineralized water, oils, emulsifiers, preservatives, sequestrants (chelating agents), fragrance and others cosmetically acceptable components.

A few examples of inert adjuvants and constituents compatible with the properties of the compositions described herein and that, additionally, may be employed in the present cosmetic composition are given hereinafter—in a non-restrictive, but only demonstrative manner
Water: water is the base of a number of preferred embodiments of the cosmetic composition of the present invention, acting as a carrier for other components. The compositions of the present invention comprise water, preferably demineralized or distilled at a suitable percentage (q.s.p.) for reaching 100% of the formula, based on the total weight of the present composition. Naturally, one may use other cosmetically acceptable carriers in the present invention;
skin conditioning agent pantenol;
emollients: olus oil, stearylic PPG-15 ether, dicapryl carbonate, silicones, cyclometicone, dimeticonol, cyclopentasyloxane, hydrogenated palm oil;
antioxidant agents: butylated hydroxidetoluene (BHT), butylated hydroxide anisol (BHA), among other;
chelating agents: EDTA, among others;
consistency agents: silica dimethyl sililate, magnesium silicate (talc), ceresin wax, hydroxypropyl starch phosphate; and
emulsifying agents: steareth-2, steareth-21, cetostearyl alcohol, ceteareth-20.

The composition according to the present invention may be present in different cosmetic forms as, for instance, and without any limitation, in the form of roll-on or cream deodorant.

According to a preferred embodiment of the present invention, the deodorant cosmetic composition of the present invention comprises:
2-methyl-5-cyclohexylpentanol in an amount ranging from 0.1 to 1% by weight, preferably from 0.3 to 0.5%, more preferably 0.4% as a deodorant active ingredient;
50% solution of aluminum hydrochloride in an amount ranging from 5.0 to 40% by weight, preferably from 10 to 35%, more preferably 30%, as an active ingredient and antiperspirant agent;
Pantenol in an amount ranging from 0.5 to 5% by weight, preferably from 0.8 to 3%, more preferably from 1 to 1.5% as a skin conditioning agent;
BHT in an amount ranging from 0.1 to 0.5% by weight, preferably from 0.04 to 0.3%, more preferably 0.05% as an antioxidant agent;
DMDM hydantoin in an amount ranging from 0.1 to 1% by weight, preferably from 0.3 to 0.8%, more preferably 0.6% as a preservative agent;
EDTA in an amount ranging from 0.05 to 0.5% by weight, preferably from 0.08 to 0.2%, more preferably 0.1% as a chelating agent;
Silica dimethyl silicate, magnesium silicate (talc), ceresin wax, hydroxypropylic starch phosphate in an amount ranging from 0.05 to 6% by weight, preferably from 0.08 to 5%, more preferably from 0.15 to 4%, as a consistency agent;
Olus soil, stearylic PPG-15 ether, hydrogenated palm oil, dicapryl carbonate, cyclomethicone silicones, dimethiconol, cyclopentasiloxane, in an amount ranging from 0.5 to 15% by weight, preferably from 0.8 to 10%, more preferably from 0.1 to 7% as emollients; and
Steareth-2, steareth-21, cetostearyl alcohol, ceteareth-20 in an amount ranging from 0.5 to 15% by weight, preferably from 1.0 to 10%, more preferably from 1.1 to 9.5% as emulsifying agents.

The antiperspirant deodorant cosmetic composition with dermo-calming action of the present invention has a number of advantages and desired characteristics with the ideal and balanced combination between its components, some of which are listed below:
differentiated antiperspirant protection;
differentiated viscosity;
differentiated hydration;
protective film characteristic;
differentiated softness; and
dermo-calming action, particularly for sensitized skins.

The embodiments of the invention exemplified hereinafter are intended to illustrate, without limiting, the scope of their object.

### EXAMPLES

### Example 1

### The Cosmetic Composition of the Present Invention in Roll-on Form (Called 12-33171-07)

Table 1 below presents an example of formulation of the cosmetic composition according to the present invention in roll-on form.

TABLE 1

| Component | Concentration (% by weight) |
|---|---|
| Demineralized water | 56.3 |
| Aluminum hydrochloride (50% solution) | 30.0 |
| Olus oil | 3.8 |
| Steareth-2 | 3.0 |
| Hydroxypropylic starch phosphate | 1.5 |
| Steareth-21 | 1.1 |
| Stearylic PPG-15 ether | 1.0 |
| Perfume | 1.0 |
| Pantenol | 1.0 |
| DMDM hydantoin | 0.6 |
| 2-methyl-5-cyclohexylpentanol | 0.4 |
| Silica dimethyl silicate | 0.15 |
| Disodium EDTA | 0.1 |
| BHT | 0.05 |

### Example 2

### The Cosmetic Composition of the Present Invention in Roll-on Form (Called 13-39540-01) without Olus Oil

Table 2 below presents one more example of formulation of the cosmetic composition according to the present invention in roll-on form.

TABLE 2

| Component | Concentration (wt.-%) |
|---|---|
| Demineralized water | 60.1 |
| Aluminum hydrochloride (50% solution) | 30.0 |
| Steareth-2 | 3.0 |
| Hydroxypropylic starch phosphate | 1.5 |
| Steareth-21 | 1.1 |
| Stearylic PPG-15 ether | 1.0 |
| Perfume | 1.0 |
| Pantenol | 1.0 |
| DMDM hidantoin | 0.6 |
| 2-methyl-5-cyclohexylpentanol | 0.4 |
| Silica dimethyl silicate | 0.15 |
| Disodium EDTA | 0.1 |
| BHT | 0.05 |

### Example 3

### The Composition of the Present Invention in Roll-On Form (Called 13-39540-02 (with Palm Oil)

Table 3 below presents one more example of formulation of the cosmetic composition according to the present invention in roll-on form.

TABLE 3

| Component | Concentration (wt.-%) |
|---|---|
| Demineralized water | 59.1 |
| Aluminum hydrochloride (50% solution) | 30.0 |
| Steareth-2 | 3.0 |
| Hydroxypropylic starch phosphate | 1.5 |
| Steareth-21 | 1.1 |
| Stearylic PPG-15 ether | 1.0 |
| Hydrogenated palm oil | 1.0 |
| Perfume | 1.0 |
| Pantenol | 1.0 |
| DMDM hydantoin | 0.6 |
| 2-methyl-5-cyclohexylpentanol | 0.4 |
| Silica dimethyl silicate | 0.15 |
| Disodium EDTA | 0.1 |
| BHT | 0.05 |

### Example 4

### The Cosmetic Composition of the Present Invention in Cream Form (Deo Cream NT1123-12-A)

Table 4 below presents a formulation of the cosmetic composition according to the present invention in cream form.

TABLE 4

| Component | Concentration (wt.-%) |
|---|---|
| Demineralized water | 41.7 |
| Aluminum hydrochloride | 30.0 |
| Cetosterayl alcohol | 7.5 |
| Olus oil | 3.8 |
| Ceresin | 3.0 |
| Cyclopentasyloxane | 2.5 |
| Talc | 2.0 |
| Hydroxypropylic starch phosphate | 2.0 |
| Steareth-20 | 1.75 |
| Pantenol | 1.5 |
| Perfume | 1.1 |
| Dicapryl carbonate | 1.0 |
| Dimethiconol, cyclopentasiloxane | 1.0 |
| DMDM hydantoin | 0.6 |
| 2-methyl-5-cyclohexylpentanol | 0.4 |
| Disodium EDTA | 0.1 |
| BHT | 0.05 |

### Example 5

### The Cosmetic Composition of the Present Invention in Cream Form (Called 13-39540-03)

Table 5 below presents a formulation of the cosmetic composition according to the present invention in cream form.

TABLE 5

| Component | Concentration (wt.-%) |
|---|---|
| Demineralized water | 45.5 |
| Aluminum hydrochloride | 30.0 |
| Cetosterayl alcohol | 7.5 |
| Ceresin | 3.0 |
| Cyclopentasiloxane | 2.5 |
| Talc | 2.0 |
| Hydroxypropylic starch phostate | 2.0 |
| Ceteareth-20 | 1.75 |
| Pantenol | 1.5 |
| Perfume | 1.1 |
| Dicapryl carbonate | 1.0 |
| Dimethiconol, cyclopeontasiloxane | 1.0 |
| DMDM hydantoin | 0.6 |
| 2.-methyl-5-cyclohexylpoentanol | 0.4 |
| Disodium EDTA | 0.1 |
| BHT | 0.05 |

### Example 6

### The Cosmetic Composition of the Present Invention in Cream Form (Called 13-39540-04)

Table 6 below presents a formulation of the cosmetic composition according to the present invention in cream form.

TABLE 6

| Component | Concentration (wt.-%) |
|---|---|
| Demineralized water | 44.5 |
| Aluminum hydrochloride | 30.0 |
| Cetostearyl alcohol | 7.5 |
| Cerasin | 3.0 |
| cyclopeontasiloxane | 2.5 |
| Talc | 2.0 |
| Hydroxypropylic starch phosphate | 2.0 |
| Ceteareth-20 | 1.75 |
| Pantenol | 1.5 |
| Perfume | 1.1 |
| Hydrogenated palm oil | 1.0 |
| Dicapryl carbonate | 1.0 |
| Dimethiconol, cyclopentasiloxane | 1.0 |
| DMDM hydantoin | 0.6 |
| 2-methyl-5-cydohexylpentanol | 0.4 |
| Disodium EDTA | 0.1 |
| BHT | 0.05 |

The cosmetic composition of the present invention is prepared in a conventional way, known to those skilled in the art.

Tests:

Test 1—Instrumental Evaluation of the Effect of the Composition of the Invention (Called 13-39540-03) on Erythema Caused by the Tape-Stripping Technique.

### 1.1—Objective

Evaluating the potential of a calming action of a topical product referring to a composition according to the invention through instrumental measurements of colorimetry.

### 1.2 Methodology, Materials and Equipment

Two symmetrical 10 cm² areas having randomized distribution in the front region of the forearms of the patients (a total of 21 participants) were demarcated. One of the areas was used for application of the product and the other was kept as control (untreated area). Colorimetric measurements were made with the equipment Mexameter MX18-Courge+Khazaka prior to tape-stripping removal (Fita Hipoalergênica Transproe^{MR}) and application of the product (Tb) after 30 tape-stripping removals at each site (T0) and after 30 minutes, 1, 2, 3, 4, 5 and 6 hours from application of the product.

### 1.3—Measurement of Erythema-Mexametry

The measurements were carried out by using the equipment Mexameter MX 18, Courage+Khazaka electronic GmbH through a measurement probe. The readings were made by applying the probe to the test areas with the pressure permitted by the spring (0.5 N).

The measurement area was 5 mm in diameter. Three measurements were carried out in each area. The measurement consisted in measuring the light absorbed and reflected at the wavelengths for green and red for hemoglobin and wavelengths for green and near infrared for melanin.

The operator positioned the probe vertically, forming a 90-degree angle with the skin and cleaned the probe with the aid of a very soft piece of paper prior to the first reading and between the readings of one area an another, even if it were the control area or the initial measurement of each area.

The reading indicated the degree of erythema of the skin. The scale of the equipment is arbitrary, the reading values indicating greater redness of the skin (erythema).

### 1.4—Steps of Research

### 1.4.1—First Step

The participants remained at rest in an air-conditioned room with temperature of 20±2° C. and relative humidity of 50±5% for at least 30 minutes prior to each reading;
The participants were told not to smoke; not to come out of the test room without prior authorization of the expert; not to come into contact with the area being tested at any place; not to contact the area being tested in contact with the clothes between the first air-conditioning and the end of the measurements; not to make abrupt movements with any part of the body; and not to allow the test area to get wet
The participants were evaluated by the dermatologist to confirm the research inclusion criteria;
Two symmetric 10 cm² areas of the region before the forearms having randomized distribution were demarcated. One area was used for application of the product and the other area was kept as negative control (untreated area); and
One determined the coloration of the areas through arithmetic mean of three measurements (Tb).

### 1.4.2—Second Step

30 tape-stripping removals were carried out in the two demarcated areas. The adhesive tapes (TransporeMR) were replaced at each removal; and
One determined the coloration of the demarcated areas through the arithmetic mean of three measurements (T0).

### 1.4.3—Third Step

The tested product was applied in a randomized manner, in the amount of 0.02 g in the demarcated region of each participant. The product was spread over the skin with the aid of a latex fingerstall, with light and circular movements until the whole application area was entirely covered and homogeneous. The latex fingerstall was changed in each area;
The measurements were carried out in the following times:
T30min—thirty minutes after application of the product
T1h—one hour after application of the product
T2h—two hours after application of the product
T3h—three hours after application of the product;
T4h—four hours after application of the product;
T5h—five hours after application of the product;
T6h—six hours after application of the product.
The participants were evaluated by the dermatologist at the end of the measurements T6h; and
After the medical evaluation the participants were released.

### 1.5—Statistical Analysis

For a statistical analysis of the results, different tests were employed, as follows:

In order to compare the treatments in each time, one used ANOVA, followed by the DUNNET test and to compare the times with regard to the initial time T0, one used the Student test t.

As said before, the number of participants of the research was 21 and all of them completed the study in question.

The trust level considered in the comparative analyses was of 96%.

### 1.6—Results

### 1.6.1—Measurement of Erythema-Mexametry

TABLE 7
Measurements, descriptive statistics and results of the comparison

| Participant of the | 13-39540-03 | | Control | | 13-39540-03 | Control |
|---|---|---|---|---|---|---|
| research | Tb | T0 | Tb | T0 | Δ(T0 − Tb) | Δ(T0 − Tb) |
| 001 | 242 | 352 | 265 | 358 | 110 | 93 |
| 002 | 343 | 493 | 363 | 478 | 150 | 115 |
| 003 | 333 | 454 | 317 | 423 | 121 | 106 |
| 004 | 251 | 385 | 272 | 399 | 134 | 127 |
| 005 | 240 | 480 | 280 | 451 | 240 | 171 |
| 006 | 451 | 551 | 395 | 465 | 100 | 70 |
| 007 | 361 | 479 | 366 | 459 | 118 | 93 |
| 008 | 342 | 450 | 354 | 448 | 108 | 94 |
| 009 | 298 | 456 | 323 | 391 | 158 | 68 |
| 010 | 357 | 452 | 401 | 467 | 95 | 66 |
| 011 | 343 | 430 | 374 | 407 | 87 | 33 |
| 012 | 276 | 299 | 264 | 323 | 23 | 59 |
| 013 | 337 | 432 | 368 | 455 | 95 | 87 |
| 014 | 362 | 452 | 346 | 448 | 90 | 102 |
| 015 | 284 | 377 | 335 | 407 | 93 | 72 |
| 017 | 312 | 477 | 316 | 405 | 165 | 89 |
| 018 | 234 | 412 | 242 | 376 | 178 | 134 |
| 019 | 282 | 357 | 306 | 374 | 75 | 68 |
| 020 | 304 | 458 | 266 | 455 | 154 | 189 |
| 021 | 267 | 360 | 267 | 336 | 93 | 69 |
| 022 | 285 | 365 | 248 | 309 | 80 | 61 |
| Average | 309.7 | 427.2 | 317.5 | 411.1 | 117.5 | 93.6 |
| Medium | 304.0 | 450.0 | 317.0 | 407.0 | 108.0 | 89.0 |
| Minimum | 234.0 | 299.0 | 242.0 | 309.0 | 23.0 | 33.0 |
| Maximum | 451.0 | 551.0 | 401.0 | 478.0 | 240.0 | 189.0 |
| Standard error | 11.5 | 13.1 | 11.0 | 11.0 | 10.0 | 8.2 |
| IC of 95% | [286.6; | [401.0; | [295.6; | [389.1; | [97.5; | [77.2; |
| | 332.8] | 453.3] | 339.5] | 433.2] | 137.4] | 110.0] |

| | | |
|---|---|---|
| Δ (%) with respect to T0 | 37.9 | 29.5 |
| % of participants with irritating effect | 100.0 | 100.0 |
| P-Value | <0.001*** | <0.001*** |

***significant at level 0.1%;
**significant at level 1%;
*significant at level 5% (Student t test)

The T0 was higher on average than the Tb for product and control.

TABLE 8
Measurements and descriptive statistics of the product

| | | | | | | | | | Δ(T30- |
|---|---|---|---|---|---|---|---|---|---|
| | | T30- | | | | | | | min − |
| Participant | T0 | min | T1 h | T2 h | T3 h | T4 h | T5 h | T6 h | T0) |
| 001 | 352 | 217 | 216 | 270 | 265 | 300 | 292 | 275 | −135 |
| 002 | 493 | 308 | 345 | 376 | 409 | 392 | 395 | 402 | −185 |
| 003 | 454 | 225 | 266 | 328 | 337 | 368 | 343 | 346 | −229 |
| 004 | 385 | 224 | 251 | 225 | 251 | 281 | 254 | 248 | −161 |
| 005 | 480 | 326 | 353 | 348 | 361 | 365 | 379 | 378 | −154 |
| 006 | 551 | 437 | 451 | 471 | 458 | 495 | 492 | 484 | −114 |
| 007 | 479 | 370 | 358 | 353 | 369 | 379 | 389 | 381 | −109 |
| 008 | 450 | 313 | 288 | 301 | 322 | 347 | 340 | 351 | −137 |
| 009 | 456 | 332 | 350 | 339 | 389 | 387 | 384 | 380 | −124 |
| 010 | 452 | 359 | 332 | 358 | 338 | 358 | 340 | 361 | −93 |
| 011 | 430 | 320 | 311 | 328 | 309 | 308 | 339 | 351 | −110 |
| 012 | 299 | 263 | 275 | 235 | 280 | 263 | 289 | 276 | −36 |
| 013 | 432 | 373 | 358 | 353 | 380 | 382 | 376 | 360 | −59 |
| 014 | 452 | 344 | 352 | 368 | 358 | 356 | 361 | 364 | −108 |
| 015 | 377 | 278 | 273 | 279 | 300 | 309 | 311 | 323 | −99 |
| 017 | 477 | 320 | 347 | 372 | 377 | 400 | 394 | 376 | −157 |
| 018 | 412 | 240 | 240 | 266 | 257 | 265 | 278 | 264 | −172 |
| 019 | 357 | 244 | 248 | 270 | 270 | 264 | 283 | 290 | −113 |
| 020 | 458 | 393 | 366 | 401 | 398 | 407 | 442 | 401 | −65 |
| 021 | 360 | 254 | 293 | 282 | 305 | 326 | 307 | 337 | −106 |
| 022 | 365 | 305 | 277 | 287 | 292 | 252 | 274 | 280 | −60 |
| Average | 427.2 | 306.9 | 311.9 | 324.3 | 334.5 | 343.0 | 345.8 | 344.2 | −120.3 |
| Medium | 450.0 | 313.0 | 311.0 | 328.0 | 337.0 | 356.0 | 340.0 | 351.0 | −113.0 |
| Minimum | 299.0 | 217.0 | 216.0 | 225.0 | 251.0 | 252.0 | 254.0 | 248.0 | −229.0 |
| Maximum | 551.0 | 437.0 | 451.0 | 471.0 | 458.0 | 495.0 | 492.0 | 484.0 | −36.0 |
| Standard | 13.1 | 13.3 | 12.3 | 13.0 | 12.3 | 13.3 | 13.1 | 12.4 | 10.1 |
| error | | | | | | | | | |
| IC de | [401.0; | [280.4; | [287.4; | [298.3; | [309.9; | [316.4; | [319.6; | [319.4; | [−140.5; |
| 95% | 453.3] | 333.4] | 336.5] | 350.2] | 359.2] | 369.7] | 372.0] | 369.0] | −100.1] |

| Δ (%) with respect to the T0 | | | | | | | −28.2 |
|---|---|---|---|---|---|---|---|
| | | Δ(T1 h − | Δ(T2 h − | Δ(T3 h − | Δ(T4 h − | Δ(T5 h − | Δ(T6 h − |
| | Participant | T0) | T0) | T0) | T0) | T0) | T0) |
| | 001 | −136 | −82 | −87 | −52 | −60 | −77 |
| | 002 | −148 | −117 | −84 | −101 | −98 | −91 |
| | 003 | −188 | −126 | −117 | −86 | −111 | −108 |
| | 004 | −134 | −160 | −134 | −104 | −131 | −137 |
| | 005 | −127 | −132 | −119 | −115 | −101 | −102 |
| | 006 | −100 | −80 | −93 | −56 | −59 | −67 |
| | 007 | −121 | −126 | −110 | −100 | −90 | −98 |
| | 008 | −162 | −149 | −128 | −103 | −110 | −99 |
| | 009 | −106 | −117 | −67 | −69 | −72 | −76 |
| | 010 | −120 | −94 | −114 | −94 | −112 | −91 |
| | 011 | −119 | −102 | −121 | −122 | −91 | −79 |
| | 012 | −24 | −64 | −19 | −36 | −10 | −23 |
| | 013 | −74 | −79 | −52 | −50 | −56 | −72 |
| | 014 | −100 | −84 | −94 | −96 | −91 | −88 |
| | 015 | −104 | −98 | −77 | −68 | −66 | −54 |
| | 017 | −130 | −105 | −100 | −77 | −83 | −101 |
| | 018 | −172 | −146 | −155 | −147 | −134 | −148 |
| | 019 | −109 | −87 | −87 | −93 | −74 | −67 |
| | 020 | −92 | −57 | −60 | −51 | −16 | −57 |
| | 021 | −67 | −78 | −55 | −34 | −53 | −23 |
| | 022 | −88 | −78 | −73 | −113 | −91 | −85 |
| | Average | −115.3 | −102.9 | −92.7 | −84.1 | −81.4 | −83.0 |
| | Medium | −119.0 | −98.0 | −93.0 | −93.0 | −90.0 | −85.0 |
| | Minimum | −188.0 | −160.0 | −155.0 | −147.0 | −134.0 | −148.0 |
| | Maximum | −24.0 | −57.0 | −19.0 | −34.0 | −10.0 | −23.0 |
| | Standard | 8.1 | 6.3 | 7.0 | 6.6 | 7.1 | 6.6 |
| | error | | | | | | |
| | IC de | [−131.4; | [−115.5; | [−106.7; | [−97.3; | [−95.5; | [−96.3; |
| | 95% | −99.2] | −90.3] | −78.6] | −71.0] | −67.2] | −69.7] |
| | Δ (%) with | −27.0 | −24.1 | −21.7 | −19.7 | −19.1 | −19.4 |
| | respect to | | | | | | |
| | the T0 | | | | | | |

TABLE 9
Descriptive measurements and statistics of the control

| | | | | | | | | | Δ(T30- |
|---|---|---|---|---|---|---|---|---|---|
| | | T30- | | | | | | | min − |
| Participant | T0 | min | T1 h | T2 h | T3 h | T4 h | T5 h | T6 h | T0) |
| 001 | 358 | 261 | 281 | 290 | 320 | 332 | 335 | 346 | −97 |
| 002 | 478 | 397 | 429 | 399 | 421 | 413 | 422 | 433 | −81 |
| 003 | 423 | 289 | 328 | 319 | 321 | 346 | 331 | 332 | −134 |
| 004 | 399 | 231 | 266 | 261 | 268 | 293 | 277 | 286 | −168 |
| 005 | 451 | 349 | 344 | 349 | 392 | 346 | 366 | 361 | −102 |
| 006 | 465 | 416 | 411 | 391 | 402 | 406 | 399 | 389 | −49 |
| 007 | 459 | 387 | 372 | 351 | 356 | 375 | 384 | 367 | −72 |
| 008 | 448 | 356 | 334 | 344 | 346 | 377 | 359 | 367 | −92 |
| 009 | 391 | 356 | 361 | 343 | 354 | 365 | 361 | 364 | −35 |
| 010 | 467 | 393 | 382 | 385 | 375 | 428 | 394 | 379 | −74 |
| 011 | 407 | 347 | 334 | 331 | 352 | 353 | 364 | 360 | −60 |
| 012 | 323 | 270 | 275 | 235 | 259 | 250 | 260 | 270 | −53 |
| 013 | 455 | 437 | 425 | 405 | 429 | 422 | 403 | 416 | −18 |
| 014 | 448 | 363 | 354 | 380 | 386 | 389 | 375 | 371 | −85 |
| 015 | 407 | 328 | 336 | 323 | 324 | 344 | 331 | 349 | −79 |
| 017 | 405 | 293 | 316 | 332 | 340 | 352 | 338 | 353 | −112 |
| 018 | 376 | 217 | 219 | 240 | 269 | 277 | 282 | 257 | −159 |
| 019 | 374 | 272 | 291 | 276 | 289 | 284 | 304 | 295 | −102 |
| 020 | 455 | 365 | 346 | 326 | 324 | 349 | 347 | 350 | −90 |
| 021 | 336 | 267 | 258 | 275 | 297 | 302 | 302 | 328 | −69 |
| 022 | 309 | 278 | 260 | 247 | 240 | 226 | 203 | 223 | −31 |
| Average | 411.1 | 327.2 | 329.6 | 323.9 | 336.4 | 344.2 | 339.9 | 342.7 | −83.9 |
| Medium | 407.0 | 347.0 | 334.0 | 331.0 | 340.0 | 349.0 | 347.0 | 353.0 | −81.0 |
| Minimum | 309.0 | 217.0 | 219.0 | 235.0 | 240.0 | 226.0 | 203.0 | 223.0 | −168.0 |
| Maximum | 478.0 | 437.0 | 429.0 | 405.0 | 429.0 | 428.0 | 422.0 | 433.0 | −18.0 |
| Standard | 11.0 | 13.7 | 12.5 | 11.6 | 11.7 | 12.1 | 11.7 | 11.2 | 8.4 |
| error | | | | | | | | | |
| IC of | [389.1; | [299.9; | [304.6; | [300.7; | [313.0; | [320; | [316.4; | [320.2; | [−100.7; |
| 95% | 433.2] | 354.6] | 354.6] | 347.1] | 359.7] | 368.5] | 363.3] | 365.1] | −67.1] |

| Δ (%) em relação ao T0 | | | | | | | −20.4 |
|---|---|---|---|---|---|---|---|
| | | Δ(T1 h − | Δ(T2 h − | Δ(T3 h − | Δ(T4 h − | Δ(T5 h − | Δ(T6 h − |
| | Participant | T0) | T0) | T0) | T0) | T0) | T0) |
| | 001 | −77 | −68 | −38 | −26 | −23 | −12 |
| | 002 | −49 | −79 | −57 | −65 | −56 | −45 |
| | 003 | −95 | −104 | −102 | −77 | −92 | −91 |
| | 004 | −133 | −138 | −131 | −106 | −122 | −113 |
| | 005 | −107 | −102 | −59 | −105 | −85 | −90 |
| | 006 | −54 | −74 | −63 | −59 | −66 | −76 |
| | 007 | −87 | −108 | −103 | −84 | −75 | −92 |
| | 008 | −114 | −104 | −102 | −71 | −89 | −81 |
| | 009 | −30 | −48 | −37 | −26 | −30 | −27 |
| | 010 | −85 | −82 | −92 | −39 | −73 | −88 |
| | 011 | −73 | −76 | −55 | −54 | −43 | −47 |
| | 012 | −48 | −88 | −64 | −73 | −63 | −53 |
| | 013 | −30 | −50 | −26 | −33 | −52 | −39 |
| | 014 | −94 | −68 | −62 | −59 | −73 | −77 |
| | 015 | −71 | −84 | −83 | −63 | −76 | −58 |
| | 017 | −89 | −73 | −65 | −53 | −67 | −52 |
| | 018 | −157 | −136 | −107 | −99 | −94 | −119 |
| | 019 | −83 | −98 | −85 | −90 | −70 | −79 |
| | 020 | −109 | −129 | −131 | −106 | −108 | −105 |
| | 021 | −78 | −61 | −39 | −34 | −34 | −8 |
| | 022 | −49 | −62 | −69 | −83 | −106 | −86 |
| | Average | −81.5 | −87.2 | −74.8 | −66.9 | −71.3 | −68.5 |
| | Medium | −83.0 | −82.0 | −65.0 | −65.0 | −73.0 | −77.0 |
| | Minimum | −157.0 | −138.0 | −131.0 | −106.0 | −122.0 | −119.0 |
| | Maximum | −30.0 | −48.0 | −26.0 | −26.0 | −23.0 | −8.0 |
| | Standard | 7.0 | 5.7 | 6.6 | 5.7 | 5.7 | 6.8 |
| | error | | | | | | |
| | IC of | [−95.6; | [−98.6; | [−87.9; | [−78.3; | [−82.7; | [−82.1; |
| | 95% | −67.5] | −75.8] | −61.7] | −55.5] | −59.9] | −54.8] |
| | Δ (%) em | −19.8 | −21.2 | −18.2 | −16.3 | −17.3 | −16.7 |
| | relação | | | | | | |
| | ao T0 | | | | | | |

TABLE 10
Average and standard error of each treatment per time, and the difference
between the treatments

| | | Product | | Control | | Product − Control | |
|---|---|---|---|---|---|---|---|
| Time | | Average | E.P. | Average | E.P. | Average | E.P. |
| T0 | | 427.19 | 13.08 | 411.14 | 11.02 | 16.05 | 7.26 |
| T30 | min | 306.90 | 13.26 | 327.24 | 13.69 | −20.33 | 7.17 |
| T1 | h | 311.90 | 12.28 | 329.62 | 12.49 | −17.71 | 8.39 |
| T2 | h | 324.29 | 12.98 | 323.90 | 11.60 | 0.38 | 7.82 |
| T3 | h | 334.52 | 12.32 | 336.38 | 11.67 | −1.86 | 8.05 |
| T4 | h | 343.05 | 13.32 | 344.24 | 12.13 | −1.19 | 8.48 |
| T5 | h | 345.81 | 13.10 | 339.86 | 11.73 | 5.95 | 9.21 |
| T6 | h | 344.19 | 12.40 | 342.67 | 11.24 | 1.52 | 8.23 |

TABLE 11
Porcentage of variation on the average with respect to the time T0,
and percentage of participants with positive and negative effect

| | | Product - Control | | |
|---|---|---|---|---|
| | | % of variation | % of participants | % of participants |
| | | with respect | with positive | with negative |
| Time | | to the T0 | effect | effect |
| T30 | min | −7.7 | 85.7 | 14.3 |
| T1 | h | −7.2 | 85.7 | 14.3 |
| T2 | h | −2.9 | 85.7 | 14.3 |
| T3 | h | −3.5 | 85.7 | 14.3 |
| T4 | h | −3.4 | 76.2 | 19.0 |
| T5 | h | −1.7 | 76.2 | 23.8 |
| T6 | h | −2.8 | 71.4 | 28.6 |

The product exhibited greater reduction with respect to the T0 in the average values of erythema compared with the control in the times T30min (p-value <0.0001), T1h (p-value=0.0001), T2h (p-value=0.0155), T3h (p-value=0.0253) and T4h (p-value=0.0163). No significant differences were found between the product and the control in the times T5h (p-value=0.2834) and T6h (p-value=0.0528).

The times T30min, T1h, T2h, T3h, T4h, T5h and T6h were lower than the time T0 for the product (p-values <0.001).

### Wherein:

cm²: square centimeters;
g: grams;
h: hours;
no.: number;
° C.: degrees Centigrade;
Tx: time after x hours of application of the product.

### 1.7—Conclusion

After the statistical analysis of the results, one can conclude that the composition of the invention promoted reduction of the erythema caused by the tape-stripping technique until the time of 4 hours.

Test 2—Instrumental Evaluation of the Effect of the Composition of the Invention (Called 13-29540-04) on the Erythema Caused by the Tape-Stripping Technique

### 2.1—Objective

Evaluating the potential of calming action of a topical product referring to a composition according to the invention through instrumental colorimetric measurement.

### 2.2—Methodology, Materials and Equipment

Two symmetric 10 cm² areas of randomized distribution in the region before the forearms of the participants (a total of 21 participants) were demarcated. One of the areas was used for application of the product and the other was kept as control (untreated area). Colorimetric measurements were made with the equipment Mexameter MX18-Courage+Khazaka before tape-stripping removal (Fita Hipoalergên Transpore^{MR}) and application of the product (Tb), after the 30 tape-stripping removals at each site (T0) and after 30 minutes, 1, 2, 3, 4, 5 and 6 hours of application of the product.

### 2.3—Measurement of Erythema-Mexametry

The measurements were carried out by using the equipment Mexameter MX 18, Courage+Khazaka electronic GmbH through a measurement probe. The readings were made by applying the probe to the test areas with the pressure permitted by the spring (0.5 N).

The measurement area was 5 mm in diameter. Three measurements were carried out in each area. The measurement consisted in measuring the light absorbed and reflected at the wavelengths for green and red for hemoglobin and wavelengths for green and near infrared for melanin.

The operator positioned the probe vertically, forming a 90-degree angle with the skin and cleaned the probe with the aid of a very soft piece of paper prior to the first reading and between the readings of one area an another, even if it were the control area or the initial measurement of each area.

The reading indicated the degree of erythema of the skin. The scale of the equipment is arbitrary, the reading values indicating greater redness of the skin (erythema).

### 2.4—Steps of Research

### 2.4.1—First Step

The participants remained at rest in an air-conditioned room with temperature of 20±2° C. and relative humidity of 50±5% for at least 30 minutes prior to each reading;
The participants were told not to smoke; not to come out of the test room without prior authorization of the expert; not to come into contact with the area being tested at any place; not to contact the area being tested in contact with the clothes between the first air-conditioning and the end of the measurements; not to make abrupt movements with any part of the body; and not to allow the test area to get wet
The participants were evaluated by the dermatologist to confirm the research inclusion criteria;
Two symmetric 10 cm² areas of the region before the forearms having randomized distribution were demarcated. One area was used for application of the product and the other area was kept as negative control (untreated area); and
One determined the coloration of the areas through arithmetic mean of three measurements (Tb).

### 2.4.2—Second Step

30 tape-stripping removals were carried out in the two demarcated areas. The adhesive tapes (TransporeMR) were replaced at each removal; and
One determined the coloration of the demarcated areas through the arithmetic mean of three measurements (T0).

### 2.4.3—Third Step

The tested product was applied in a randomized manner, in the amount of 0.02 g in the demarcated region of each participant. The product was spread over the skin with the aid of a latex fingerstall, with light and circular movements until the whole application area was entirely covered and homogeneous. The latex fingerstall was changed in each area;
The measurements were carried out in the following times:
T30min—thirty minutes after application of the product
T1h—one hour after application of the product
T2h—two hours after application of the product
T3h—three hours after application of the product;
T4h—four hours after application of the product;
T5h—five hours after application of the product;
T6h—six hours after application of the product.
The participants were evaluated by the dermatologist at the end of the measurements T6h; and
After the medical evaluation the participants were released.

### 2.5—Statistical Analysis

For a statistical analysis of the results, different tests were employed, as follows:

In order to compare the treatments in each time, one used ANOVA, followed by the DUNNET test and to compare the times with regard to the initial time T0, one used the Student test t.

As said before, the number of participants of the research was 21 and all of them completed the study in question.

The trust level considered in the comparative analyses was of 96%.

### 2.6—Results

### 2.6.1—Measurement of Erythema-Mexametry

TABLE 12
Measurements, descriptive statistics and results of the comparison

Participant

| of the | 13-39540-04 | | Control | | 13-39540-04 | Control |
|---|---|---|---|---|---|---|
| research | Tb | T0 | Tb | T0 | Δ(T0 − Tb) | Δ(T0 − Tb) |
| 001 | 279 | 374 | 265 | 358 | 95 | 93 |
| 002 | 300 | 426 | 363 | 478 | 126 | 115 |
| 003 | 303 | 428 | 317 | 423 | 125 | 106 |
| 004 | 264 | 395 | 272 | 399 | 131 | 127 |
| 005 | 236 | 412 | 280 | 451 | 176 | 171 |
| 006 | 403 | 505 | 395 | 465 | 102 | 70 |
| 007 | 403 | 484 | 366 | 459 | 81 | 93 |
| 008 | 358 | 442 | 354 | 448 | 84 | 94 |
| 009 | 309 | 428 | 323 | 391 | 119 | 68 |
| 010 | 383 | 459 | 401 | 467 | 76 | 66 |
| 011 | 350 | 417 | 374 | 407 | 67 | 33 |
| 012 | 313 | 389 | 264 | 323 | 76 | 59 |
| 013 | 360 | 407 | 368 | 455 | 47 | 87 |
| 014 | 326 | 388 | 346 | 448 | 62 | 102 |
| 015 | 302 | 398 | 335 | 407 | 96 | 72 |
| 017 | 304 | 344 | 316 | 405 | 40 | 89 |
| 018 | 244 | 423 | 242 | 376 | 179 | 134 |
| 019 | 279 | 383 | 306 | 374 | 104 | 68 |
| 020 | 306 | 410 | 266 | 455 | 104 | 189 |
| 021 | 220 | 371 | 267 | 336 | 151 | 69 |
| 022 | 270 | 301 | 248 | 309 | 31 | 61 |
| Average | 310.1 | 408.8 | 317.5 | 411.1 | 98.7 | 93.6 |
| Medium | 304.0 | 410.0 | 317.0 | 407.0 | 96.0 | 89.0 |
| Minimum | 220.0 | 301.0 | 242.0 | 309.0 | 31.0 | 33.0 |
| Maximum | 403.0 | 505.0 | 401.0 | 478.0 | 179.0 | 189.0 |
| Standard | 11.2 | 9.8 | 11.0 | 11.0 | 8.8 | 8.2 |
| error | | | | | | |
| IC of 95% | [287.6; 332.6] | [389.2; 428.3] | [295.6; 339.5] | [389.1; 433.2] | [81.0; 116.3] | [77.2; 110.0] |

| | | |
|---|---|---|
| Δ(%) with respect to the T0 | 31.8 | 29.5 |
| % of participants with irritating effect | 100.0 | 100.0 |
| P-Value | <0.001*** | <0.001*** |

***significant at level 0.1%;
**significant at level 1%;
*significant at level 5% (t-Student test).

The T0 was hither on the average than the Tb for product and control

TABLE 13
Measurements and descriptive statistics of the product

| Partici- | | T30- | | | | | | |
|---|---|---|---|---|---|---|---|---|
| pant | T0 | min | T1 h | T2 h | T3 h | T4 h | T5 h | T6 h |
| 001 | 374 | 235 | 257 | 280 | 295 | 334 | 331 | 346 |
| 002 | 426 | 284 | 263 | 323 | 341 | 328 | 347 | 331 |
| 003 | 428 | 234 | 266 | 276 | 314 | 340 | 328 | 317 |
| 004 | 395 | 246 | 276 | 267 | 270 | 295 | 293 | 290 |
| 005 | 412 | 263 | 273 | 307 | 298 | 304 | 285 | 289 |
| 006 | 505 | 380 | 390 | 408 | 423 | 441 | 424 | 428 |
| 007 | 484 | 391 | 388 | 380 | 385 | 389 | 398 | 394 |
| 008 | 442 | 312 | 304 | 275 | 313 | 319 | 321 | 332 |
| 009 | 428 | 321 | 336 | 331 | 353 | 401 | 344 | 371 |
| 010 | 459 | 369 | 375 | 388 | 392 | 416 | 417 | 386 |
| 011 | 417 | 355 | 323 | 335 | 350 | 350 | 386 | 353 |
| 012 | 389 | 324 | 305 | 295 | 294 | 311 | 290 | 306 |
| 013 | 407 | 362 | 373 | 342 | 390 | 371 | 354 | 378 |
| 014 | 388 | 292 | 315 | 330 | 319 | 317 | 297 | 306 |
| 015 | 398 | 300 | 311 | 309 | 314 | 332 | 311 | 313 |
| 017 | 344 | 257 | 282 | 280 | 307 | 321 | 303 | 311 |
| 018 | 423 | 260 | 264 | 283 | 284 | 300 | 290 | 290 |
| 019 | 383 | 254 | 259 | 258 | 275 | 280 | 294 | 275 |
| 020 | 410 | 345 | 317 | 312 | 359 | 348 | 335 | 349 |
| 021 | 371 | 226 | 244 | 264 | 270 | 260 | 271 | 284 |
| 022 | 301 | 251 | 244 | 245 | 251 | 222 | 213 | 227 |
| Average | 408.8 | 298.1 | 303.1 | 309.0 | 323.7 | 332.3 | 325.3 | 327.4 |
| Medium | 410.0 | 292.0 | 304.0 | 307.0 | 314.0 | 328.0 | 321.0 | 317.0 |
| Minimum | 301.0 | 226.0 | 244.0 | 245.0 | 251.0 | 222.0 | 213.0 | 227.0 |
| Maximum | 505.0 | 391.0 | 390.0 | 408.0 | 423.0 | 441.0 | 424.0 | 428.0 |
| Standard | 9.8 | 11.6 | 10.3 | 9.7 | 10.2 | 11.3 | 11.1 | 10.3 |
| error | | | | | | | | |
| IC of 95% | [389.2; 428.3] | [275.0; 321.2] | [282.5; 323.6] | [289.6; 328.3] | [303.3; 344.1] | [309.8; 354.9] | [303.1; 347.6] | [306.9; 348.0] |

Δ(%) em relação ao T0

| | Δ(T30- | Δ | Δ | Δ | Δ | Δ | Δ |
|---|---|---|---|---|---|---|---|
| Partici- | min − | (T1 h − | (T2 h − | (T3 h − | (T4 h − | (T5 h − | (T6 h − |
| pant | T0) | T0) | T0) | T0) | T0) | T0) | T0) |
| 001 | −139 | −117 | −94 | −79 | −40 | −43 | −28 |
| 002 | −142 | −163 | −103 | −85 | −98 | −79 | −95 |
| 003 | −194 | −162 | −152 | −114 | −88 | −100 | −111 |
| 004 | −149 | −119 | −128 | −125 | −100 | −102 | −105 |
| 005 | −149 | −139 | −105 | −114 | −108 | −127 | −123 |
| 006 | −125 | −115 | −97 | −82 | −64 | −81 | −77 |
| 007 | −93 | −96 | −104 | −99 | −95 | −86 | −90 |
| 008 | −130 | −138 | −167 | −129 | −123 | −121 | −110 |
| 009 | −107 | −92 | −97 | −75 | −27 | −84 | −57 |
| 010 | −90 | −84 | −71 | −67 | −43 | −42 | −73 |
| 011 | −62 | −94 | −82 | −67 | −67 | −31 | −64 |
| 012 | −65 | −84 | −94 | −95 | −78 | −99 | −83 |
| 013 | −45 | −34 | −65 | −17 | −36 | −53 | −29 |
| 014 | −96 | −73 | −58 | −69 | −71 | −91 | −82 |
| 015 | −98 | −87 | −89 | −84 | −66 | −87 | −85 |
| 017 | −87 | −62 | −64 | −37 | −23 | −41 | −33 |
| 018 | −163 | −159 | −140 | −139 | −123 | −133 | −133 |
| 019 | −129 | −124 | −125 | −108 | −103 | −89 | −108 |
| 020 | −65 | −93 | −98 | −51 | −62 | −75 | −61 |
| 021 | −145 | −127 | −107 | −101 | −111 | −100 | −87 |
| 022 | −50 | −57 | −56 | −50 | −79 | −88 | −74 |
| Average | −110.6 | −105.7 | −99.8 | −85.1 | −76.4 | −83.4 | −81.3 |
| Medium | −107.0 | −96.0 | −97.0 | −84.0 | −78.0 | −87.0 | −83.0 |
| Minimum | −194.0 | −163.0 | −167.0 | −139.0 | −123.0 | −133.0 | −133.0 |
| Maximum | −45.0 | −34.0 | −56.0 | −17.0 | −23.0 | −31.0 | −28.0 |
| Standard | 8.8 | 7.7 | 6.5 | 6.8 | 6.7 | 6.2 | 6.4 |
| error | | | | | | | |
| IC of 95% | [−128.3; −93.0] | [−121.1; −90.2] | [−112.9; −86.8] | [−98.7; −71.4] | [−89.8; −63.1] | [−95.8; −71.1] | [−94.1; −68.6] |
| Δ(%) em | −27.1 | −25.9 | −24.4 | −20.8 | −18.7 | −20.4 | −19.9 |
| relação | | | | | | | |
| ao T0 | | | | | | | |

TABLE 14
Measurements and descriptive statistics of the control

| Partici- | | T30- | | | | | | |
|---|---|---|---|---|---|---|---|---|
| pant | T0 | min | T1 h | T2 h | T3 h | T4 h | T5 h | T6 h |
| 001 | 358 | 261 | 281 | 290 | 320 | 332 | 335 | 346 |
| 002 | 478 | 397 | 429 | 399 | 421 | 413 | 422 | 433 |
| 003 | 423 | 289 | 328 | 319 | 321 | 346 | 331 | 332 |
| 004 | 399 | 231 | 266 | 261 | 268 | 293 | 277 | 286 |
| 005 | 451 | 349 | 344 | 349 | 392 | 346 | 366 | 361 |
| 006 | 465 | 416 | 411 | 391 | 402 | 406 | 399 | 389 |
| 007 | 459 | 387 | 372 | 351 | 356 | 375 | 384 | 367 |
| 008 | 448 | 356 | 334 | 344 | 346 | 377 | 359 | 367 |
| 009 | 391 | 356 | 361 | 343 | 354 | 365 | 361 | 364 |
| 010 | 467 | 393 | 382 | 385 | 375 | 428 | 394 | 379 |
| 011 | 407 | 347 | 334 | 331 | 352 | 353 | 364 | 360 |
| 012 | 323 | 270 | 275 | 235 | 259 | 250 | 260 | 270 |
| 013 | 455 | 437 | 425 | 405 | 429 | 422 | 403 | 416 |
| 014 | 448 | 363 | 354 | 380 | 386 | 389 | 375 | 371 |
| 015 | 407 | 328 | 336 | 323 | 324 | 344 | 331 | 349 |
| 017 | 405 | 293 | 316 | 332 | 340 | 352 | 338 | 353 |
| 018 | 376 | 217 | 219 | 240 | 269 | 277 | 282 | 257 |
| 019 | 374 | 272 | 291 | 276 | 289 | 284 | 304 | 295 |
| 020 | 455 | 365 | 346 | 326 | 324 | 349 | 347 | 350 |
| 021 | 336 | 267 | 258 | 275 | 297 | 302 | 302 | 328 |
| 022 | 309 | 278 | 260 | 247 | 240 | 226 | 203 | 223 |
| Average | 411.1 | 327.2 | 329.6 | 323.9 | 336.4 | 344.2 | 339.9 | 342.7 |
| Medium | 407.0 | 347.0 | 334.0 | 331.0 | 340.0 | 349.0 | 347.0 | 353.0 |
| Minimum | 309.0 | 217.0 | 219.0 | 235.0 | 240.0 | 226.0 | 203.0 | 223.0 |
| Maximum | 478.0 | 437.0 | 429.0 | 405.0 | 429.0 | 428.0 | 422.0 | 433.0 |
| Standard | 11.0 | 13.7 | 12.5 | 11.6 | 11.7 | 12.1 | 11.7 | 11.2 |
| error | | | | | | | | |
| IC of 95% | [389.1; 433.2] | [299.9; 354.6] | [304.6; 354.6] | [300.7; 347.1] | [313.0; 359.7] | [320; 368.5] | [316.4; 363.3] | [320.2; 365.1] |

Δ(%) wtih respect to the T0

| | Δ(T30- | Δ | Δ | Δ | Δ | Δ | Δ |
|---|---|---|---|---|---|---|---|
| Partici- | min − | (T1 h − | (T2 h − | (T3 h − | (T4 h − | (T5 h − | (T6 h − |
| pant | T0) | T0) | T0) | T0) | T0) | T0) | T0) |
| 001 | −97 | −77 | −68 | −38 | −26 | −23 | −12 |
| 002 | −81 | −49 | −79 | −57 | −65 | −56 | −45 |
| 003 | −134 | −95 | −104 | −102 | −77 | −92 | −91 |
| 004 | −168 | −133 | −138 | −131 | −106 | −122 | −113 |
| 005 | −102 | −107 | −102 | −59 | −105 | −85 | −90 |
| 006 | −49 | −54 | −74 | −63 | −59 | −66 | −76 |
| 007 | −72 | −87 | −108 | −103 | −84 | −75 | −92 |
| 008 | −92 | −114 | −104 | −102 | −71 | −89 | −81 |
| 009 | −35 | −30 | −48 | −37 | −26 | −30 | −27 |
| 010 | −74 | −85 | −82 | −92 | −39 | −73 | −88 |
| 011 | −60 | −73 | −76 | −55 | −54 | −43 | −47 |
| 012 | −53 | −48 | −88 | −64 | −73 | −63 | −53 |
| 013 | −18 | −30 | −50 | −26 | −33 | −52 | −39 |
| 014 | −85 | −94 | −68 | −62 | −59 | −73 | −77 |
| 015 | −79 | −71 | −84 | −83 | −63 | −76 | −58 |
| 017 | −112 | −89 | −73 | −65 | −53 | −67 | −52 |
| 018 | −159 | −157 | −136 | −107 | −99 | −94 | −119 |
| 019 | −102 | −83 | −98 | −85 | −90 | −70 | −79 |
| 020 | −90 | −109 | −129 | −131 | −106 | −108 | −105 |
| 021 | −69 | −78 | −61 | −39 | −34 | −34 | −8 |
| 022 | −31 | −49 | −62 | −69 | −83 | −106 | −86 |
| Average | −83.9 | −81.5 | −87.2 | −74.8 | −66.9 | −71.3 | −68.5 |
| Medium | −81.0 | −83.0 | −82.0 | −65.0 | −65.0 | −73.0 | −77.0 |
| Minimum | −168.0 | −157.0 | −138.0 | −131.0 | −106.0 | −122.0 | −119.0 |
| Maximum | −18.0 | −30.0 | −48.0 | −26.0 | −26.0 | −23.0 | −8.0 |
| Standard | 8.4 | 7.0 | 5.7 | 6.6 | 5.7 | 5.7 | 6.8 |
| error | | | | | | | |
| IC of 95% | [−100.7; −67.1] | [−95.6; −67.5] | [−98.6; −75.8] | [−87.9; −61.7] | [−78.3; −55.5] | [−82.7; −59.9] | [−82.1; −54.8] |
| Δ(%) wtih | −20.4 | −19.8 | −21.2 | −18.2 | −16.3 | −17.3 | −16.7 |
| respect | | | | | | | |
| to the T0 | | | | | | | |

TABLE 15
Average and standard error of each treatment per time and the difference
between the treatments

| | | Product | | Control | | Product − Cotrol | |
|---|---|---|---|---|---|---|---|
| Time | | Average | E.P. | Average | E.P. | Average | E.P. |
| T0 | | 408.76 | 9.77 | 411.14 | 11.02 | −2.38 | 8.14 |
| T30 | min | 298.14 | 11.55 | 327.24 | 13.69 | −29.10 | 8.74 |
| T1 | h | 303.10 | 10.28 | 329.62 | 12.49 | −26.52 | 9.26 |
| T2 | h | 308.95 | 9.67 | 323.90 | 11.60 | −14.95 | 7.92 |
| T3 | h | 323.67 | 10.21 | 336.38 | 11.67 | −12.71 | 7.88 |
| T4 | h | 332.33 | 11.26 | 344.24 | 12.13 | −11.90 | 8.05 |
| T5 | h | 325.33 | 11.14 | 339.86 | 11.73 | −14.52 | 7.56 |
| T6 | h | 327.43 | 10.29 | 342.67 | 11.24 | −15.24 | 8.17 |

TABLE 16
Porcentage of variation on the average with respect to the time T0,
and percentage of participants with positive and negative effect

| | | Product - Control | | |
|---|---|---|---|---|
| | | % of variation | % of subjects | % of subjects |
| | | with respect | with positive | with negative |
| Time | | to the T0 | effect | effect |
| T30 | min | −6.7 | 85.7 | 14.3 |
| T1 | h | −6.0 | 76.2 | 23.8 |
| T2 | h | −3.2 | 66.7 | 33.3 |
| T3 | h | −2.6 | 66.7 | 33.3 |
| T4 | h | −2.4 | 81.0 | 19.0 |
| T5 | h | −3.1 | 71.4 | 28.6 |
| T6 | h | −3.2 | 66.7 | 33.3 |

The product exhibited greater reduction with respect to the T0 in the average values of erythema compared with the control in the times T30min (p-value=0.0004) and T1h (p-value=0.0031). No significant differences were found between product and control in the times T2h (p-value=0.0571), T3h (p-value=o.0571), T3h (p-value=0.2448), T4h (p-value=0.2311), T5h (p-value=0.1731) and T6h (p-value=0.0925).

The times T30min, T1h, T2h, T3h, T4h, T5h and T6h were lower that the T0 for the product (p-values <0.001);

wherein:
cm2: square centimeters;
g: grams;
h: hours;
no.: number;
° C.: degrees Centigrade;
Tx: Time after x hours of application of the product.

### 2.7—Conclusion

After a statistic analysis of the results, one can conclude that the composition of the invention promoted reduction of the erythema caused by the tape-stripping technique until the time of 1 hour.

Test 3—Instrumental Evaluation of the Effect of the Composition of the Invention (Called 13-39540-01) on the Erythema Caused by the Tape-Stripping Technique

### 3.1—Objective

Evaluating the potential of a calming action of a topical product referring to a composition according to the invention through instrumental measurements of colorimetry.

3.2—Methodology. Materials and Equipment

Two symmetrical 10 cm² areas having randomized distribution in the front region of the forearms of the patients (a total of 21 participants) were demarcated. One of the areas was used for application of the product and the other was kept as control (untreated area). Colorimetric measurements were made with the equipment Mexameter MX18-Couage+Khazaka prior to tape-stripping removal (Fita Hipoalergênica Transproe^{MR}) and application of the product (Tb) after 30 tape-stripping removals at each site (T0) and after 30 minutes, 1, 2, 3, 4, 5 and 6 hours from application of the product.

### 3.3—Measurement of Erythema-Mexametry

The measurements were carried out by using the equipment Mexameter MX 18, Courage+Khazaka electronic GmbH through a measurement probe. The readings were made by applying the probe to the test areas with the pressure permitted by the spring (0.5 N).

The measurement area was 5 mm in diameter. Three measurements were carried out in each area. The measurement consisted in measuring the light absorbed and reflected at the wavelengths for green and red for hemoglobin and wavelengths for green and near infrared for melanin.

The operator positioned the probe vertically, forming a 90-degree angle with the skin and cleaned the probe with the aid of a very soft piece of paper prior to the first reading and between the readings of one area an another, even if it were the control area or the initial measurement of each area.

The reading indicated the degree of erythema of the skin. The scale of the equipment is arbitrary, the reading values indicating greater redness of the skin (erythema).

### 3.4—Steps of Research

### 3.4.1—First Step

The participants remained at rest in an air-conditioned room with temperature of 20±2° C. and relative humidity of 50±5% for at least 30 minutes prior to each reading;
The participants were told not to smoke; not to come out of the test room without prior authorization of the expert; not to come into contact with the area being tested at any place; not to contact the area being tested in contact with the clothes between the first air-conditioning and the end of the measurements; not to make abrupt movements with any part of the body; and not to allow the test area to get wet
The participants were evaluated by the dermatologist to confirm the research inclusion criteria;
Two symmetric 10 cm² areas of the region before the forearms having randomized distribution were demarcated. One area was used for application of the product and the other area was kept as negative control (untreated area); and
One determined the coloration of the areas through arithmetic mean of three measurements (Tb).

### 3.4.2—Second Step

30 tape-stripping removals were carried out in the two demarcated areas. The adhesive tapes (TransporeMR) were replaced at each removal; and
One determined the coloration of the demarcated areas through the arithmetic mean of three measurements (T0).

### 3.4.3—Third Step

The tested product was applied in a randomized manner, in the amount of 0.02 g in the demarcated region of each participant. The product was spread over the skin with the aid of a latex fingerstall, with light and circular movements until the whole application area was entirely covered and homogeneous. The latex fingerstall was changed in each area;
The measurements were carried out in the following times:
T30min—thirty minutes after application of the product
T1h—one hour after application of the product
T2h—two hours after application of the product
T3h—three hours after application of the product;
T4h—four hours after application of the product;
T5h—five hours after application of the product;
T6h—six hours after application of the product.
The participants were evaluated by the dermatologist at the end of the measurements T6h; and
After the medical evaluation the participants were released.

### 3.5—Statistical Analysis

For a statistical analysis of the results, different tests were employed, as follows:

In order to compare the treatments in each time, one used ANOVA, followed by the DUNNET test and to compare the times with regard to the initial time T0, one used the Student test t.

As said before, the number of participants of the research was 21 and all of them completed the study in question.

The trust level considered in the comparative analyses was of 96%.

### 3.6—Results

### 3.6.1—Measurement of Erythema-Mexametry

TABLE 17
Measurements, descriptive statistics and results of the comparison

| Participant da | 13-39540-01 | | Control | | 13-39540-01 | Control |
|---|---|---|---|---|---|---|
| pesquisa | Tb | T0 | Tb | T0 | Δ (T0 − Tb) | Δ (T0 − Tb) |
| 001 | 338 | 428 | 334 | 428 | 90 | 94 |
| 002 | 252 | 400 | 278 | 401 | 148 | 123 |
| 003 | 221 | 354 | 207 | 291 | 133 | 84 |
| 004 | 292 | 391 | 269 | 396 | 99 | 127 |
| 005 | 326 | 433 | 334 | 389 | 107 | 55 |
| 006 | 176 | 323 | 178 | 334 | 147 | 156 |
| 007 | 365 | 479 | 342 | 451 | 114 | 109 |
| 008 | 303 | 397 | 268 | 393 | 94 | 125 |
| 009 | 311 | 421 | 339 | 420 | 110 | 81 |
| 010 | 245 | 398 | 248 | 391 | 153 | 143 |
| 011 | 209 | 356 | 238 | 350 | 147 | 112 |
| 012 | 230 | 363 | 247 | 361 | 133 | 114 |
| 013 | 278 | 383 | 304 | 486 | 105 | 182 |
| 014 | 340 | 415 | 347 | 436 | 75 | 89 |
| 015 | 237 | 318 | 225 | 322 | 81 | 97 |
| 016 | 228 | 352 | 258 | 368 | 124 | 110 |
| 017 | 270 | 381 | 234 | 339 | 111 | 105 |
| 018 | 284 | 411 | 311 | 406 | 127 | 95 |
| 019 | 226 | 318 | 231 | 318 | 92 | 87 |
| 020 | 288 | 425 | 263 | 372 | 137 | 109 |
| 021 | 300 | 362 | 292 | 365 | 62 | 73 |
| Average | 272.3 | 386.1 | 273.7 | 381.8 | 113.8 | 108.1 |
| Medium | 278.0 | 391.0 | 268.0 | 389.0 | 111.0 | 109.0 |
| Minimum | 176.0 | 318.0 | 178.0 | 291.0 | 62.0 | 55.0 |
| Maximum | 365.0 | 479.0 | 347.0 | 486.0 | 153.0 | 182.0 |
| Standard error | 10.8 | 9.1 | 10.5 | 10.4 | 5.7 | 6.3 |
| IC of 95% | [250.8; 293.8] | [367.9; 404.3] | [252.6; 294.8] | [360.9; 402.6] | [102.4; 125.1] | [95.5; 120.7] |

| | | |
|---|---|---|
| Δ (%) with respect to the T0 | 41.8 | 39.5 |
| % of participants with irritating effect | 100.0 | 100.0 |
| P-Value | <0.001*** | <0.001*** |

***significant at level 0.1%;
**significant at level 1%;
*significant at level 5% (t-Student test).

The T0 was higher on the average than the Tb for product and control

TABLE 18
Measurements and descriptive statistics of the product

| | | | | | | | | | Δ (T30- |
|---|---|---|---|---|---|---|---|---|---|
| | | T30- | | | | | | | min − |
| Participant | T0 | min | T1 h | T2 h | T3 h | T4 h | T5 h | T6 h | T0) |
| 001 | 428 | 331 | 345 | 371 | 371 | 366 | 376 | 364 | −97 |
| 002 | 400 | 270 | 300 | 320 | 320 | 308 | 304 | 317 | −130 |
| 003 | 354 | 247 | 260 | 232 | 261 | 209 | 219 | 234 | −107 |
| 004 | 391 | 273 | 288 | 313 | 328 | 312 | 321 | 322 | −118 |
| 005 | 433 | 298 | 305 | 303 | 302 | 314 | 336 | 346 | −135 |
| 006 | 323 | 154 | 159 | 167 | 164 | 168 | 172 | 210 | −169 |
| 007 | 479 | 328 | 325 | 300 | 360 | 386 | 376 | 389 | −151 |
| 008 | 397 | 263 | 279 | 292 | 277 | 297 | 309 | 291 | −134 |
| 009 | 421 | 302 | 312 | 326 | 336 | 332 | 351 | 339 | −119 |
| 010 | 398 | 282 | 283 | 304 | 305 | 302 | 298 | 303 | −116 |
| 011 | 356 | 232 | 234 | 241 | 245 | 252 | 276 | 257 | −124 |
| 012 | 363 | 244 | 234 | 227 | 254 | 268 | 242 | 255 | −119 |
| 013 | 383 | 288 | 283 | 303 | 306 | 325 | 319 | 335 | −95 |
| 014 | 415 | 353 | 337 | 363 | 379 | 360 | 384 | 392 | −62 |
| 015 | 318 | 287 | 277 | 291 | 291 | 300 | 307 | 319 | −31 |
| 016 | 352 | 213 | 202 | 217 | 224 | 254 | 244 | 293 | −139 |
| 017 | 381 | 280 | 318 | 317 | 315 | 319 | 321 | 326 | −101 |
| 018 | 411 | 284 | 270 | 280 | 287 | 324 | 328 | 356 | −127 |
| 019 | 318 | 230 | 226 | 239 | 272 | 255 | 264 | 260 | −88 |
| 020 | 425 | 310 | 300 | 302 | 297 | 291 | 345 | 345 | −115 |
| 021 | 362 | 328 | 317 | 320 | 356 | 353 | 333 | 352 | −34 |
| Average | 386.1 | 276.0 | 278.8 | 287.0 | 297.6 | 299.8 | 306.0 | 314.5 | −110.0 |
| Medium | 391.0 | 282.0 | 283.0 | 302.0 | 302.0 | 308.0 | 319.0 | 322.0 | −118.0 |
| Minimum | 318.0 | 154.0 | 159.0 | 167.0 | 164.0 | 168.0 | 172.0 | 210.0 | −169.0 |
| Maximum | 479.0 | 353.0 | 345.0 | 371.0 | 379.0 | 386.0 | 384.0 | 392.0 | −31.0 |
| Standard | 9.1 | 10.0 | 10.1 | 10.9 | 11.3 | 11.4 | 11.8 | 10.8 | 7.5 |
| error | | | | | | | | | |
| IC of | [367.9; | [256; | [258.5; | [265.3; | [275.1; | [277; | [282.4; | [293; | [−125.1; |
| 95% | 404.3] | 296.1] | 299] | 308.8] | 320.1] | 322.5] | 329.5] | 336.1] | −95] |

| | |
|---|---|
| Δ (%) with respect to the T0 | −28.5 |

| | Δ | Δ | Δ | Δ | Δ | Δ |
|---|---|---|---|---|---|---|
| | (T1 h − | (T2 h − | (T3 h − | (T4 h − | (T5 h − | (T6 h − |
| Participant | T0) | T0) | T0) | T0) | T0) | T0) |
| 001 | −83 | −57 | −57 | −62 | −52 | −64 |
| 002 | −100 | −80 | −80 | −92 | −96 | −83 |
| 003 | −94 | −122 | −93 | −145 | −135 | −120 |
| 004 | −103 | −78 | −63 | −79 | −70 | −69 |
| 005 | −128 | −130 | −131 | −119 | −97 | −87 |
| 006 | −164 | −156 | −159 | −155 | −151 | −113 |
| 007 | −154 | −179 | −119 | −93 | −103 | −90 |
| 008 | −118 | −105 | −120 | −100 | −88 | −106 |
| 009 | −109 | −95 | −85 | −89 | −70 | −82 |
| 010 | −115 | −94 | −93 | −96 | −100 | −95 |
| 011 | −122 | −115 | −111 | −104 | −80 | −99 |
| 012 | −129 | −136 | −109 | −95 | −121 | −108 |
| 013 | −100 | −80 | −77 | −58 | −64 | −48 |
| 014 | −78 | −52 | −36 | −55 | −31 | −23 |
| 015 | −41 | −27 | −27 | −18 | −11 | 1 |
| 016 | −150 | −135 | −128 | −98 | −108 | −59 |
| 017 | −63 | −64 | −66 | −62 | −60 | −55 |
| 018 | −141 | −131 | −124 | −87 | −83 | −55 |
| 019 | −92 | −79 | −46 | −63 | −54 | −58 |
| 020 | −125 | −123 | −128 | −134 | −80 | −80 |
| 021 | −45 | −42 | −6 | −9 | −29 | −10 |
| Average | −107.3 | −99.0 | −88.5 | −86.3 | −80.1 | −71.6 |
| Medium | −109.0 | −95.0 | −93.0 | −92.0 | −80.0 | −80.0 |
| Minimum | −164.0 | −179.0 | −159.0 | −155.0 | −151.0 | −120.0 |
| Maximum | −41.0 | −27.0 | −6.0 | −9.0 | −11.0 | 1.0 |
| Standard | 7.3 | 8.6 | 8.7 | 8.0 | 7.6 | 7.2 |
| error | | | | | | |
| IC of | [−122; | [−116.2; | [−105.8; | [−102.2; | [−95.3; | [−85.9; −57.2] |
| 95% | −92.8] | −81.9] | −71.1] | −70.4] | −65] | |
| Δ (%) with respect to the | −27.8 | −25.7 | −22.9 | −22.4 | −20.8 | −18.5 |
| T0 | | | | | | |

TABLE 19
Measurements and descriptive statistics of the control

| | | | | | | | | | Δ (T30- |
|---|---|---|---|---|---|---|---|---|---|
| | | T30- | | | | | | | min − |
| Participant | T0 | min | T1 h | T2 h | T3 h | T4 h | T5 h | T6 h | T0) |
| 001 | 428 | 340 | 351 | 382 | 381 | 384 | 357 | 379 | −88 |
| 002 | 401 | 326 | 329 | 337 | 338 | 323 | 333 | 331 | −75 |
| 003 | 291 | 245 | 243 | 250 | 249 | 246 | 241 | 234 | −46 |
| 004 | 396 | 283 | 315 | 339 | 360 | 352 | 343 | 369 | −113 |
| 005 | 389 | 325 | 324 | 331 | 319 | 333 | 349 | 344 | −64 |
| 006 | 334 | 180 | 229 | 203 | 214 | 248 | 251 | 260 | −154 |
| 007 | 451 | 323 | 360 | 337 | 349 | 382 | 307 | 348 | −128 |
| 008 | 393 | 288 | 296 | 308 | 292 | 294 | 287 | 321 | −105 |
| 009 | 420 | 361 | 343 | 348 | 368 | 365 | 398 | 383 | −59 |
| 010 | 391 | 281 | 306 | 325 | 326 | 325 | 323 | 343 | −110 |
| 011 | 350 | 268 | 267 | 262 | 285 | 293 | 306 | 310 | −82 |
| 012 | 361 | 270 | 292 | 261 | 312 | 282 | 287 | 301 | −91 |
| 013 | 486 | 366 | 377 | 388 | 385 | 399 | 392 | 408 | −120 |
| 014 | 436 | 396 | 397 | 412 | 421 | 416 | 432 | 419 | −40 |
| 015 | 322 | 270 | 277 | 262 | 267 | 275 | 296 | 294 | −52 |
| 016 | 368 | 244 | 222 | 241 | 248 | 264 | 262 | 301 | −124 |
| 017 | 339 | 288 | 293 | 310 | 303 | 301 | 305 | 296 | −51 |
| 018 | 406 | 319 | 313 | 318 | 312 | 344 | 316 | 338 | −87 |
| 019 | 318 | 245 | 225 | 256 | 260 | 260 | 270 | 276 | −73 |
| 020 | 372 | 293 | 290 | 279 | 331 | 316 | 348 | 357 | −79 |
| 021 | 365 | 305 | 296 | 278 | 308 | 313 | 329 | 316 | −60 |
| Average | 381.8 | 296.0 | 302.1 | 306.0 | 315.6 | 319.8 | 320.6 | 329.9 | −85.8 |
| Medium | 389.0 | 288.0 | 296.0 | 310.0 | 312.0 | 316.0 | 316.0 | 331.0 | −82.0 |
| Minimum | 291.0 | 180.0 | 222.0 | 203.0 | 214.0 | 246.0 | 241.0 | 234.0 | −154.0 |
| Maximum | 486.0 | 396.0 | 397.0 | 412.0 | 421.0 | 416.0 | 432.0 | 419.0 | −40.0 |
| Standard | 10.4 | 10.7 | 10.6 | 11.7 | 11.3 | 10.9 | 10.7 | 10.2 | 6.7 |
| error | | | | | | | | | |
| IC of | [360.9; | [274.6; | [281; | [282.6; | [293.1; | [298; | [299.2; | [309.4; | [−99.2; |
| 95% | 402.6] | 317.4] | 323.3] | 329.5] | 338.2] | 341.6] | 341.9] | 350.4] | −72.3] |

| | |
|---|---|
| Δ (%) with respect to the T0 | −22.5 |

| | Δ | Δ | Δ | Δ | Δ | Δ |
|---|---|---|---|---|---|---|
| | (T1 h − | (T2 h − | (T3 h − | (T4 h − | (T5 h − | (T6 h − |
| Participant | T0) | T0) | T0) | T0) | T0) | T0) |
| 001 | −77 | −46 | −47 | −44 | −71 | −49 |
| 002 | −72 | −64 | −63 | −78 | −68 | −70 |
| 003 | −48 | −41 | −42 | −45 | −50 | −57 |
| 004 | −81 | −57 | −36 | −44 | −53 | −27 |
| 005 | −65 | −58 | −70 | −56 | −40 | −45 |
| 006 | −105 | −131 | −120 | −86 | −83 | −74 |
| 007 | −91 | −114 | −102 | −69 | −144 | −103 |
| 008 | −97 | −85 | −101 | −99 | −106 | −72 |
| 009 | −77 | −72 | −52 | −55 | −22 | −37 |
| 010 | −85 | −66 | −65 | −66 | −68 | −48 |
| 011 | −83 | −88 | −65 | −57 | −44 | −40 |
| 012 | −69 | −100 | −49 | −79 | −74 | −60 |
| 013 | −109 | −98 | −101 | −87 | −94 | −78 |
| 014 | −39 | −24 | −15 | −20 | −4 | −17 |
| 015 | −45 | −60 | −55 | −47 | −26 | −28 |
| 016 | −146 | −127 | −120 | −104 | −106 | −67 |
| 017 | −46 | −29 | −36 | −38 | −34 | −43 |
| 018 | −93 | −88 | −94 | −62 | −90 | −68 |
| 019 | −93 | −62 | −58 | −58 | −48 | −42 |
| 020 | −82 | −93 | −41 | −56 | −24 | −15 |
| 021 | −69 | −87 | −57 | −52 | −36 | −49 |
| Average | −79.6 | −75.7 | −66.1 | −62.0 | −61.2 | −51.9 |
| Medium | −81.0 | −72.0 | −58.0 | −57.0 | −53.0 | −49.0 |
| Minimum | −146.0 | −131.0 | −120.0 | −104.0 | −144.0 | −103.0 |
| Maximum | −39.0 | −24.0 | −15.0 | −20.0 | −4.0 | −15.0 |
| Standard | 5.4 | 6.4 | 6.4 | 4.6 | 7.5 | 4.8 |
| error | | | | | | |
| IC of | [−90.4; | [−88.6; | [−78.9; −53.4] | [−71.1; −52.9] | [−76.2; | [−61.4; −42.4] |
| 95% | −68.8] | −62.9] | | | −46.2] | |
| Δ (%) with | −20.9 | −19.8 | −17.3 | −16.2 | −16.0 | −13.6 |
| respect to the T0 | | | | | | |

TABLE 20
Average and standard error of each treatment per time,
and of the difference between the treatments

| | Product | | Control | | Product − Control | |
|---|---|---|---|---|---|---|
| Time | Average | E.P. | Average | E.P. | Average | E.P. |
| T0 | 386.10 | 9.10 | 381.76 | 10.42 | 4.33 | 7.32 |
| T30 min | 276.05 | 10.01 | 296.00 | 10.71 | −19.95 | 5.77 |
| T1 h | 278.76 | 10.14 | 302.14 | 10.58 | −23.38 | 6.72 |
| T2 h | 287.05 | 10.88 | 306.05 | 11.70 | −19.00 | 6.01 |
| T3 h | 297.62 | 11.25 | 315.62 | 11.28 | −18.00 | 6.53 |
| T4 h | 299.76 | 11.38 | 319.76 | 10.90 | −20.00 | 6.49 |
| T5 h | 305.95 | 11.79 | 320.57 | 10.67 | −14.62 | 7.50 |
| T6 h | 314.52 | 10.76 | 329.90 | 10.24 | −15.38 | 7.05 |

TABLE 21
Porcentage of variation in the average with respect to the time
T0, and percentage of participants with positive and negative effect

| | | Produto-Controle | | |
|---|---|---|---|---|
| | | % of | % of | % of |
| | | variation with | participants with | participants with |
| Time | | respect to the T0 | positive effect | negative effect |
| T30 | min | −6.0 | 85.7 | 14.3 |
| T1 | h | −6.9 | 81.0 | 19.0 |
| T2 | h | −5.8 | 85.7 | 14.3 |
| T3 | h | −5.6 | 81.0 | 19.0 |
| T4 | h | −6.1 | 81.0 | 19.0 |
| T5 | h | −4.7 | 66.7 | 33.3 |
| T6 | h | −5.0 | 71.4 | 28.6 |

The product exhibited greater reduction with respect to the T0 in the average values of erythema compared with the control in the times T30min (p-value=0.0010). T1h (p-value=0.0001), T2h (p-value=0.0008), T3h (p-value=0.0050), T4h (p-value=0.0020), T5h (p-value=0.0136) and T6h (p-value=0.0031).

The times T30min, T1h, T3h, T4h, T5h and T6h were lower than the time T0 for the product (p-values <0.001).

### Wherein:

Cm²: square centimeters;
G: grams;
H: hours;
No.: number;

### ° C.: Degrees Celsius;

Tx: Time after x hours of application of the product.

### 6.4—Conclusion

After a statistic analysis of the results, one can conclude that the composition of the invention promoted reduction of the erythema caused by the tape-stripping technique until the time of 6 hours.

Test 4—Instrumental Evaluation of the Effect of the Composition of the Invention (Called 13-39540-02) on the Erythema Caused by the Tape-Stripping Technique

### 4.1—Objective

TABLE 22
Measurements. descriptive statistics and results of the comparison

| Participant of | 13-39540-02 | | Control | | 13-39540-02 | Control |
|---|---|---|---|---|---|---|
| the research | Tb | T0 | Tb | T0 | Δ (T0 − Tb) | Δ (T0 − Tb) |
| 001 | 318 | 425 | 334 | 428 | 107 | 94 |
| 002 | 278 | 391 | 278 | 401 | 113 | 123 |
| 003 | 211 | 290 | 207 | 291 | 79 | 84 |
| 004 | 308 | 436 | 269 | 396 | 128 | 127 |
| 005 | 348 | 455 | 334 | 389 | 107 | 55 |
| 006 | 168 | 326 | 178 | 334 | 158 | 156 |
| 007 | 326 | 473 | 342 | 451 | 147 | 109 |
| 008 | 304 | 379 | 268 | 393 | 75 | 125 |
| 009 | 317 | 407 | 339 | 420 | 90 | 81 |
| 010 | 253 | 383 | 248 | 391 | 130 | 143 |
| 011 | 279 | 364 | 238 | 350 | 85 | 112 |
| 012 | 265 | 379 | 247 | 361 | 114 | 114 |
| 013 | 318 | 435 | 304 | 486 | 117 | 182 |
| 014 | 325 | 418 | 347 | 436 | 93 | 89 |
| 015 | 233 | 319 | 225 | 322 | 86 | 97 |
| 016 | 236 | 349 | 258 | 368 | 113 | 110 |
| 017 | 232 | 314 | 234 | 339 | 82 | 105 |
| 018 | 336 | 447 | 311 | 406 | 111 | 95 |
| 019 | 227 | 303 | 231 | 318 | 76 | 87 |
| 020 | 291 | 395 | 263 | 372 | 104 | 109 |
| 021 | 318 | 399 | 292 | 365 | 81 | 73 |
| Average | 280.5 | 385.1 | 273.7 | 381.8 | 104.6 | 108.1 |
| Medium | 291.0 | 391.0 | 268.0 | 389.0 | 107.0 | 109.0 |
| Minimum | 168.0 | 290.0 | 178.0 | 291.0 | 75.0 | 55.0 |
| Maximum | 348.0 | 473.0 | 347.0 | 486.0 | 158.0 | 182.0 |
| Standard error | 10.5 | 11.5 | 10.5 | 10.4 | 5.1 | 6.3 |
| IC of 95% | [259.5; 301.6] | [362.1; 408.1] | [252.6; 294.8] | [360.9; 402.6] | [94.4; 114.7] | [95.5; 120.7] |

| | | |
|---|---|---|
| Δ (%) with respect to the T0 | 37.3 | 39.5 |
| % of participants with irritaging effect | 100.0 | 100.0 |
| P-Value | <0.001*** | <0.001*** |

***significant at level 0.1%;
**significant at level 1%;
*significant at level 5% (t-Student test).

The T0 was higher on the average than the Tb for product and control

TABLE 23
Measurements and descriptive statistics of the product

| | | | | | | | | | Δ (T30- |
|---|---|---|---|---|---|---|---|---|---|
| | | T30- | | | | | | | min − |
| Participant | T0 | min | T1 h | T2 h | T3 h | T4 h | T5 h | T6 h | T0) |
| 001 | 425 | 326 | 358 | 363 | 396 | 373 | 372 | 359 | −99 |
| 002 | 391 | 246 | 262 | 292 | 290 | 292 | 286 | 294 | −145 |
| 003 | 290 | 216 | 193 | 196 | 213 | 195 | 192 | 202 | −74 |
| 004 | 436 | 313 | 345 | 386 | 384 | 378 | 360 | 396 | −123 |
| 005 | 455 | 290 | 301 | 297 | 303 | 310 | 336 | 364 | −165 |
| 006 | 326 | 162 | 180 | 180 | 172 | 228 | 217 | 215 | −164 |
| 007 | 473 | 315 | 337 | 312 | 325 | 355 | 327 | 360 | −158 |
| 008 | 379 | 280 | 288 | 294 | 292 | 299 | 298 | 307 | −99 |
| 009 | 407 | 312 | 324 | 319 | 326 | 328 | 337 | 326 | −95 |
| 010 | 383 | 274 | 296 | 304 | 319 | 324 | 322 | 301 | −109 |
| 011 | 364 | 284 | 271 | 271 | 281 | 279 | 289 | 289 | −80 |
| 012 | 379 | 228 | 228 | 260 | 248 | 269 | 266 | 273 | −151 |
| 013 | 435 | 391 | 390 | 405 | 408 | 415 | 415 | 421 | −44 |
| 014 | 418 | 370 | 354 | 372 | 400 | 383 | 380 | 399 | −48 |
| 015 | 319 | 293 | 281 | 274 | 290 | 275 | 281 | 297 | −26 |
| 016 | 349 | 201 | 189 | 219 | 229 | 247 | 248 | 282 | −148 |
| 017 | 314 | 248 | 238 | 269 | 272 | 264 | 270 | 269 | −66 |
| 018 | 447 | 333 | 317 | 338 | 325 | 339 | 336 | 389 | −114 |
| 019 | 303 | 216 | 209 | 224 | 243 | 241 | 253 | 259 | −87 |
| 020 | 395 | 313 | 290 | 299 | 338 | 304 | 317 | 336 | −82 |
| 021 | 399 | 355 | 347 | 339 | 386 | 379 | 353 | 381 | −44 |
| Averate | 385.1 | 284.1 | 285.6 | 295.9 | 306.7 | 308.4 | 307.4 | 320.0 | −101.0 |
| Medium | 391.0 | 290.0 | 290.0 | 297.0 | 303.0 | 304.0 | 317.0 | 307.0 | −99.0 |
| Minimum | 290.0 | 162.0 | 180.0 | 180.0 | 172.0 | 195.0 | 192.0 | 202.0 | −165.0 |
| Maximum | 473.0 | 391.0 | 390.0 | 405.0 | 408.0 | 415.0 | 415.0 | 421.0 | −26.0 |
| Standard | 11.5 | 12.8 | 13.4 | 13.1 | 14.2 | 12.7 | 12.1 | 13.2 | 9.3 |
| error | | | | | | | | | |
| IC of | [362.1; | [258.5; | [258.9; | [269.6; | [278.3; | [282.9; | [283.1; | [293.5; | [−119.7; |
| 95% | 408.1] | 309.7] | 312.3] | 322.1] | 335] | 333.9] | 331.6] | 346.4] | −82.3] |

| | |
|---|---|
| Δ (%) with respect to the T0 | −26.2 |

| | Δ | Δ | Δ | Δ | Δ | Δ |
|---|---|---|---|---|---|---|
| | (T1 h − | (T2 h − | (T3 h − | (T4 h − | (T5 h − | (T6 h − |
| Participant | T0) | T0) | T0) | T0) | T0) | T0) |
| 001 | −67 | −62 | −29 | −52 | −53 | −66 |
| 002 | −129 | −99 | −101 | −99 | −105 | −97 |
| 003 | −97 | −94 | −77 | −95 | −98 | −88 |
| 004 | −91 | −50 | −52 | −58 | −76 | −40 |
| 005 | −154 | −158 | −152 | −145 | −119 | −91 |
| 006 | −146 | −146 | −154 | −98 | −109 | −111 |
| 007 | −136 | −161 | −148 | −118 | −146 | −113 |
| 008 | −91 | −85 | −87 | −80 | −81 | −72 |
| 009 | −83 | −88 | −81 | −79 | −70 | −81 |
| 010 | −87 | −79 | −64 | −59 | −61 | −82 |
| 011 | −93 | −93 | −83 | −85 | −75 | −75 |
| 012 | −151 | −119 | −131 | −110 | −113 | −106 |
| 013 | −45 | −30 | −27 | −20 | −20 | −14 |
| 014 | −64 | −46 | −18 | −35 | −38 | −19 |
| 015 | −38 | −45 | −29 | −44 | −38 | −22 |
| 016 | −160 | −130 | −120 | −102 | −101 | −67 |
| 017 | −76 | −45 | −42 | −50 | −44 | −45 |
| 018 | −130 | −109 | −122 | −108 | −111 | −58 |
| 019 | −94 | −79 | −60 | −62 | −50 | −44 |
| 020 | −105 | −96 | −57 | −91 | −78 | −59 |
| 021 | −52 | −60 | −13 | −20 | −46 | −18 |
| Averate | −99.5 | −89.2 | −78.4 | −76.7 | −77.7 | −65.1 |
| Medium | −93.0 | −88.0 | −77.0 | −80.0 | −76.0 | −67.0 |
| Minimum | −160.0 | −161.0 | −154.0 | −145.0 | −146.0 | −113.0 |
| Maximum | −38.0 | −30.0 | −13.0 | −20.0 | −20.0 | −14.0 |
| Standard | 8.0 | 8.3 | 9.9 | 7.3 | 7.2 | 6.8 |
| error | | | | | | |
| IC of | [−115.6; | [−105.8; | [−98.3; −58.6] | [−91.2; −62.2] | [−92.1; | [−78.7; −51.6] |
| 95% | −83.4] | −72.7] | | | −63.3] | |
| Δ (%) with | −25.8 | −23.2 | −20.4 | −19.9 | −20.2 | −16.9 |
| respect to the | | | | | | |
| T0 | | | | | | |

TABLE 24
Measurements and descriptive statistics of the control

| | | | | | | | | | Δ (T30- |
|---|---|---|---|---|---|---|---|---|---|
| | | T30- | | | | | | | min − |
| Participant | T0 | min | T1 h | T2 h | T3 h | T4 h | T5 h | T6 h | T0) |
| 001 | 428 | 340 | 351 | 382 | 381 | 384 | 357 | 379 | −88 |
| 002 | 401 | 326 | 329 | 337 | 338 | 323 | 333 | 331 | −75 |
| 003 | 291 | 245 | 243 | 250 | 249 | 246 | 241 | 234 | −46 |
| 004 | 396 | 283 | 315 | 339 | 360 | 352 | 343 | 369 | −113 |
| 005 | 389 | 325 | 324 | 331 | 319 | 333 | 349 | 344 | −64 |
| 006 | 334 | 180 | 229 | 203 | 214 | 248 | 251 | 260 | −154 |
| 007 | 451 | 323 | 360 | 337 | 349 | 382 | 307 | 348 | −128 |
| 008 | 393 | 288 | 296 | 308 | 292 | 294 | 287 | 321 | −105 |
| 009 | 420 | 361 | 343 | 348 | 368 | 365 | 398 | 383 | −59 |
| 010 | 391 | 281 | 306 | 325 | 326 | 325 | 323 | 343 | −110 |
| 011 | 350 | 268 | 267 | 262 | 285 | 293 | 306 | 310 | −82 |
| 012 | 361 | 270 | 292 | 261 | 312 | 282 | 287 | 301 | −91 |
| 013 | 486 | 366 | 377 | 388 | 385 | 399 | 392 | 408 | −120 |
| 014 | 436 | 396 | 397 | 412 | 421 | 416 | 432 | 419 | −40 |
| 015 | 322 | 270 | 277 | 262 | 267 | 275 | 296 | 294 | −52 |
| 016 | 368 | 244 | 222 | 241 | 248 | 264 | 262 | 301 | −124 |
| 017 | 339 | 288 | 293 | 310 | 303 | 301 | 305 | 296 | −51 |
| 018 | 406 | 319 | 313 | 318 | 312 | 344 | 316 | 338 | −87 |
| 019 | 318 | 245 | 225 | 256 | 260 | 260 | 270 | 276 | −73 |
| 020 | 372 | 293 | 290 | 279 | 331 | 316 | 348 | 357 | −79 |
| 021 | 365 | 305 | 296 | 278 | 308 | 313 | 329 | 316 | −60 |
| Average | 381.8 | 296.0 | 302.1 | 306.0 | 315.6 | 319.8 | 320.6 | 329.9 | −85.8 |
| Medium | 389.0 | 288.0 | 296.0 | 310.0 | 312.0 | 316.0 | 316.0 | 331.0 | −82.0 |
| Minimum | 291.0 | 180.0 | 222.0 | 203.0 | 214.0 | 246.0 | 241.0 | 234.0 | −154.0 |
| Maximum | 486.0 | 396.0 | 397.0 | 412.0 | 421.0 | 416.0 | 432.0 | 419.0 | −40.0 |
| Standard | 10.4 | 10.7 | 10.6 | 11.7 | 11.3 | 10.9 | 10.7 | 10.2 | 6.7 |
| error | | | | | | | | | |
| IC of | [360.9; | [274.6; | [281; | [282.6; | [293.1; | [298; | [299.2; | [309.4; | [−99.2; |
| 95% | 402.6] | 317.4] | 323.3] | 329.5] | 338.2] | 341.6] | 341.9] | 350.4] | −72.3] |

| | |
|---|---|
| Δ (%) with respect to the T0 | −22.5 |

| | Δ | Δ | Δ | Δ | Δ | Δ |
|---|---|---|---|---|---|---|
| | (T1 h − | (T2 h − | (T3 h − | (T4 h − | (T5 h − | (T6 h − |
| Participant | T0) | T0) | T0) | T0) | T0) | T0) |
| 001 | −77 | −46 | −47 | −44 | −71 | −49 |
| 002 | −72 | −64 | −63 | −78 | −68 | −70 |
| 003 | −48 | −41 | −42 | −45 | −50 | −57 |
| 004 | −81 | −57 | −36 | −44 | −53 | −27 |
| 005 | −65 | −58 | −70 | −56 | −40 | −45 |
| 006 | −105 | −131 | −120 | −86 | −83 | −74 |
| 007 | −91 | −114 | −102 | −69 | −144 | −103 |
| 008 | −97 | −85 | −101 | −99 | −106 | −72 |
| 009 | −77 | −72 | −52 | −55 | −22 | −37 |
| 010 | −85 | −66 | −65 | −66 | −68 | −48 |
| 011 | −83 | −88 | −65 | −57 | −44 | −40 |
| 012 | −69 | −100 | −49 | −79 | −74 | −60 |
| 013 | −109 | −98 | −101 | −87 | −94 | −78 |
| 014 | −39 | −24 | −15 | −20 | −4 | −17 |
| 015 | −45 | −60 | −55 | −47 | −26 | −28 |
| 016 | −146 | −127 | −120 | −104 | −106 | −67 |
| 017 | −46 | −29 | −36 | −38 | −34 | −43 |
| 018 | −93 | −88 | −94 | −62 | −90 | −68 |
| 019 | −93 | −62 | −58 | −58 | −48 | −42 |
| 020 | −82 | −93 | −41 | −56 | −24 | −15 |
| 021 | −69 | −87 | −57 | −52 | −36 | −49 |
| Average | −79.6 | −75.7 | −66.1 | −62.0 | −61.2 | −51.9 |
| Medium | −81.0 | −72.0 | −58.0 | −57.0 | −53.0 | −49.0 |
| Minimum | −146.0 | −131.0 | −120.0 | −104.0 | −144.0 | −103.0 |
| Maximum | −39.0 | −24.0 | −15.0 | −20.0 | −4.0 | −15.0 |
| Standard | 5.4 | 6.4 | 6.4 | 4.6 | 7.5 | 4.8 |
| error | | | | | | |
| IC of | [−90.4; | [−88.6; | [−78.9; −53.4] | [−71.1; −52.9] | [−76.2; | [−61.4; −42.4] |
| 95% | −68.8] | −62.9] | | | −46.2] | |
| Δ (%) with | −20.9 | −19.8 | −17.3 | −16.2 | −16.0 | −13.6 |
| respect to the | | | | | | |
| T0 | | | | | | |

TABLE 25
Average and standard error of each treatment per time,
and of the difference between the treatments

| | Producut | | Control | | Product − Control | |
|---|---|---|---|---|---|---|
| Time | Average | E.P. | Average | E.P. | Average | E.P. |
| T0 | 385.10 | 11.50 | 381.76 | 10.42 | 3.33 | 5.94 |
| T30 min | 284.10 | 12.78 | 296.00 | 10.71 | −11.90 | 7.02 |
| T1 h | 285.62 | 13.35 | 302.14 | 10.58 | −16.52 | 6.78 |
| T2 h | 295.86 | 13.13 | 306.05 | 11.70 | −10.19 | 6.72 |
| T3 h | 306.67 | 14.18 | 315.62 | 11.28 | −8.95 | 7.01 |
| T4 h | 308.43 | 12.74 | 319.76 | 10.90 | −11.33 | 5.56 |
| T5 h | 307.38 | 12.12 | 320.57 | 10.67 | −13.19 | 5.95 |
| T6 h | 319.95 | 13.24 | 329.90 | 10.24 | −9.95 | 6.84 |

TABLE 26
Porcentage of variation in the average with respect to the time T0,
and percentage of participants with positive and negative effect

| | | Product - Control | | |
|---|---|---|---|---|
| | | % of variation | % of participants | % of participants |
| | | with respect | with positive | with negative |
| Time | | to the T0 | effect | effect |
| T30 | min | −3.8 | 71.4 | 28.6 |
| T1 | h | −5.0 | 76.2 | 23.8 |
| T2 | h | −3.3 | 76.2 | 19.0 |
| T3 | h | −3.0 | 66.7 | 28.6 |
| T4 | h | −3.7 | 71.4 | 28.6 |
| T5 | h | −4.2 | 76.2 | 23.8 |
| T6 | h | −3.3 | 71.4 | 19.0 |

The product exhibited greater reduction with respect to the T0 in the average values of erythema compared with the control in the times T30min (p-value=0.0416), T1h (p-value=0.0040), and T5h (p-value=0.332). No significant differences were found between product and control in the times T2h (p-value=0.0579), T3h (p-value=0.1489). T4h (p-value 0.0712), and T6h (p-value=0.0508).

The times T30, T1h, T2h, T3h, T4h, T5h and T6h were lower than the time T0 for the product (p-values<0.001).

### Wherein:

Cm²: square centigrade:
G: grams;
H: hours;
No.: number;
° C.=degrees Celsius;
Tx: Time after x hours of application of the product.

### 6.5—Conclusion

After a statistical analysis of the results, one can conclude that the composition of the invention promoted reduction of the erythema caused by the tape-stripping technique until the time of 1 hour and in the time of 5 hours.

Test 5—Evaluation of the Reduction of Erythema Caused by the Tape-Stripping Technique by Using the Composition of the Invention (called NT1123-12-A).

### 5.1 Objective

Evaluating the efficacy of the topical product (called BDP 1160.18704.6) referring to a composition according to the in reducing skin erythema induced by mechanical insult and evaluated as a function of the redness of the skin. Ion this study, one evaluated the efficacy of a composition according to the invention (NT1123-12-A) in reducing cutaneous erythema with respect to a control (site without application of any products). The evaluations were carried in the following times:

t0: right after formation of the erythema induced by the tape-stripping method;

t0.5: 30 minutes after induction of the erythema and application or non-application of the product;

t1: 1 hour after induction of the erythema and application or non-application of the product;

t2: 2 hours after induction of the erythema and application or non-application of the product;

t3: 3 hours after induction of the erythema and application or non-application of the product;

t4: 4 hours after induction of the erythema and application or non-application of the product;

t5: 5 hours after induction of the erythema and application or non-application of the product.

### 5.2—Methodology, Materials and Equipment

On each voluntary woman (a total of 25 participants), two 2.5×4.0 cm rectangles were marked with a surgical pen, called sites, on the left or right forearm. After 30 minutes of air-conditioning, the environment controlled at 20±° C. and 50±5% of relative humidity of the air, 30 tape-stripping removals with Transpore® 3M tape were carried out at each site, followed by the measurements of erythema by the mexametry and colorimetry technique. These measurements were called basal measurements (t0). After the mechanical insult, the product was applied, and the voluntary women remained at the laboratory for the measurements after 0.5 (30 minutes), 1, 2, 3, 4, 5 and 6 hours. During the whole experiment the climatic conditions were kept constant according to the ranges cited before. At the site intended for the product, 80 μL of the sample under study was applied, one of the sites being used as control (without application of any products).

### 5.3—Acquisition of Measurements

The measurements were made by using a Mexameter® MX 18 probe, coupled to the equipment Multi Probe Adapter MPA-5 (CKeletronics, Germany). Concomitantly with the measurements, one used an automatized spreadsheet of the software Microsoft® Office Excel 2010 for calculation of the Variation Coefficient (VC/CV) of the readings obtained. One carried out 5 measurements per site in each time. If in 5 measurements the VC/CV presented a value lower than 4%, one would finish the measurements at the site and continue them in the next one; otherwise, the process was restarted by using the colorimeter Byk-Gardner Spectro-Guide Sphere Gloss. For this evaluation, one recorded only the values of a*, which is the axis that ranges from green (−120) to read (+120). The values were recorded on an automatized spreadsheet of the software Microsoft® Office Excel 2010 for calculation of the Coefficient of Variation (CV/VC) of the readings obtained. One carried out 3 measurements per site in each time. If in 3 measurements the CV/VC presented a value lower than 4%, one would finish the measurements at the site and continue the measurements at the next one; otherwise the process was restarted until a CV/VC lower than 4% would be obtained.

### 5.4—Results

### 5.4.1—Evaluation of the Reduction of the Erythema by Mexametry

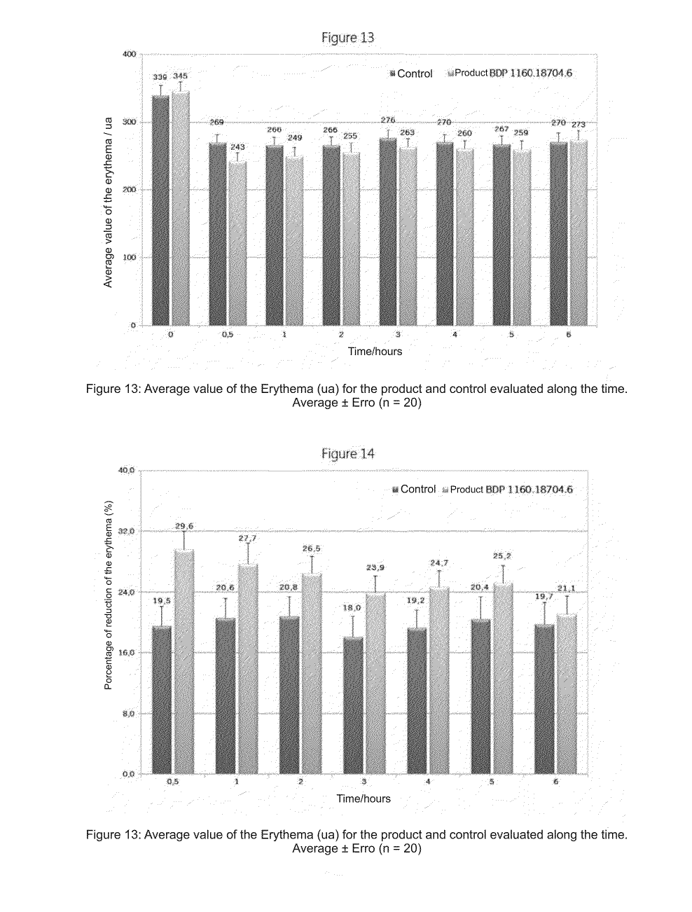

In evaluating of the reduction of the erythema by mexametry, one measured the erythema values (E) for each voluntary woman, at the site intended for the product and at the control site. FIG. 13 shows the average values of Erythema (E) obtained for the product site and control site.

In order to evaluate the homogeneity of t erythema caused by the mechanical insult, the values of erythema measured at each site in the basal time (t0) were statistically compared with each other by using the t-Student test, in pairs, with trust interval of 95%. The result is summarized in Table 27.

TABLE 27
Comparison of the values of erythema induced
after mechanical insult - Value of P.

| | |
|---|---|
| Eₜ₀ product vs. Eₜ₀ control | 0.5533 (non-significant) |

According to the results obtained, there was no statistically significant difference between the sites (P>0.05), indicating the homogeneity in the values of erythema after the mechanical insult. This indicated that the study was conducted with homogeneous basal values of erythema at the evaluation sites.

In order to evaluate whether there were significant variations in the erythema along the study, the erythema measurements obtained after 0.5; 1; 2; 3; 4; 5 and 6 hours from application of the product, or the basal measurements (t0) were not compared, carried out right after the mechanical insult (prior to application of the product), by using the single-factor variance analysis method, with Dunnett post-test multiple comparison, considering α=0.05. The results of the statistical analysis are summarized in Table 28.

TABLE 28
Summarized data of the statistical analysis. Comparison of the values
of erythema formed after the mechanical insult (t0) vs ti (wherein
i = 0.5, 1, 2, 3, 4, 5 and 6 hours from application). P values

| Comparison group | Control | NT1123-12-A |
|---|---|---|
| Eₜ₀ vs. E_{t0.5} | P < 0.05 (significant) | P < 0.05 (significant) |
| Eₜ₀ vs. Eₜ₁ | P < 0.05 (significant) | P < 0.05 (significant) |
| Eₜ₀ vs. Eₜ₂ | P < 0.05 (significant) | P < 0.05 (significant) |
| Eₜ₀ vs. Eₜ₃ | P < 0.05 (significant) | P < 0.05 (significant) |
| Eₜ₀ vs. Eₜ₄ | P < 0.05 (significant) | P < 0.05 (significant) |
| Eₜ₀ vs. Eₜ₅ | P < 0.05 (significant) | P < 0.05 (significant) |
| Eₜ₀ vs. Eₜ₆ | P < 0.05 (significant) | P < 0.05 (significant) |

According to the results, it was possible to find out that there was a statistically significant reduction (P<0.05) of the skin erythema after 0.5, 1, 2, 3, 4, 5 and h hours from application of the composition according to the invention as compared with the initial erythema (t0) and still that at the control site (without application of any products) was also obtained one observed a significant reduction (P<0.05) in the erythema formed by the mechanical insult after 0.5, 1, 2, 3, 4, 5 and 6 hours.

Although one has observed a reduction of the erythema at both sites along the study, it is possible to observe, in FIG. 14, which shows the percentage of Reduction of the Erythema (% RE) obtained in each evaluation time, what there was a more marked reduction of the erythema at the site intended for the product.

In order to evaluate the significance of the reduction of the erythema along the time, that is, the Calming Effect (EC/CE) provided by the product with respect to the control, one calculated the ratio between the erythema measurements after 0.5, 1, 2, 3, 4, 5 and 6 hours with respect to the value obtained right after the mechanical insult (t0), by using Equation 1.

EC=Eₜᵢ/Eₜ₀

Equation 1. Calculation of the Calming Effect (EC). Eₜᵢ=measurement of the coloration of the erythema in the time i (i=0.5, 1, 2, 3, 4, 5 and 6h) and Eₜ₀=measurement of the coloration of the basal erythema (right after formation).

The values of the Calming Effect obtained for the site with application of the product, in each time, were statistically compared with the values obtained for the control by using the t-Student test, bimodal, in pairs, with trust interval of 95%. The results of the statistical analysis are listed in Attachment VII and are summarized in Table 29.

TABLE 29
Comparison of the results in terms of EC between the product and the respective control.
P. values

| Comparison | | | | | | | |
|---|---|---|---|---|---|---|---|
| group | t0.5 | t1 | t2 | t3 | t4 | t5 | t6 |
| EC_{NT1123-12-A} | 0.0014 | 0.0281 | 0.0563 | 0.0787 | 0.0528 | 0.1682 | 0.6979 |
| vs. EC_{Control} | (significant) | (significant) | (Non- | (Non- | (Non- | (Non- | (Non- |
| | | | significant) | significant) | significant) | significant) | significant) |

The reduction of the skin erythema provided by the composition of the invention presented a statistically significant difference (P<0.05) after 30 minutes and 1 hour from application as compared with the respective control (site without application of any products). This indicated that the product was effective in reducing skin erythema by mechanical insult and evaluated as a function of the redness of the skin until 1 hour after application.

### 5.4.2—Evaluation of the Reduction of the Erythema by Colorimetry

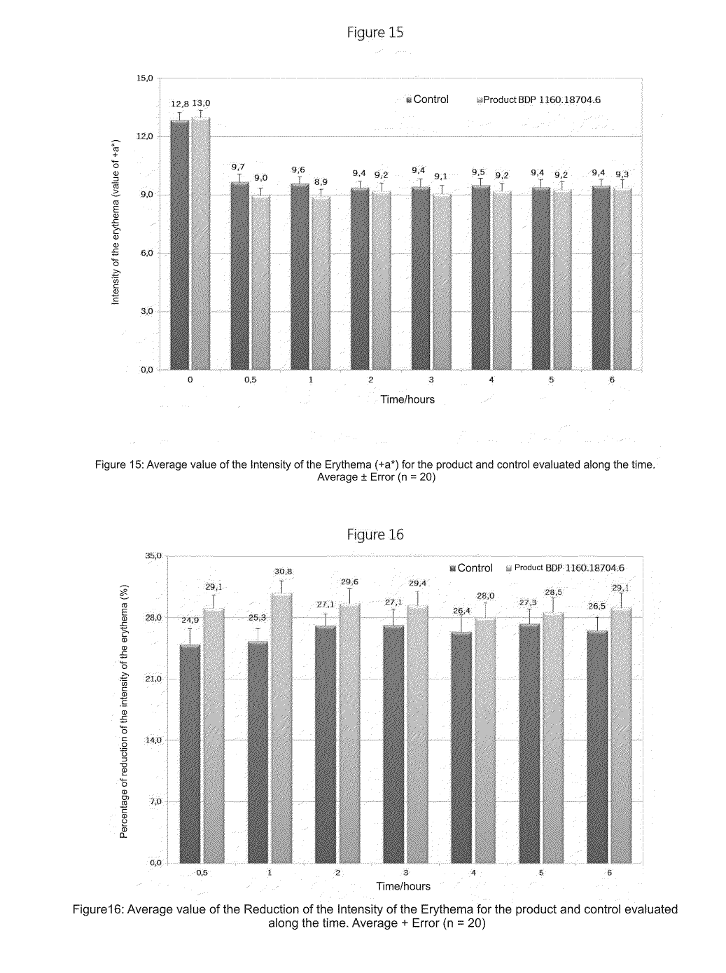

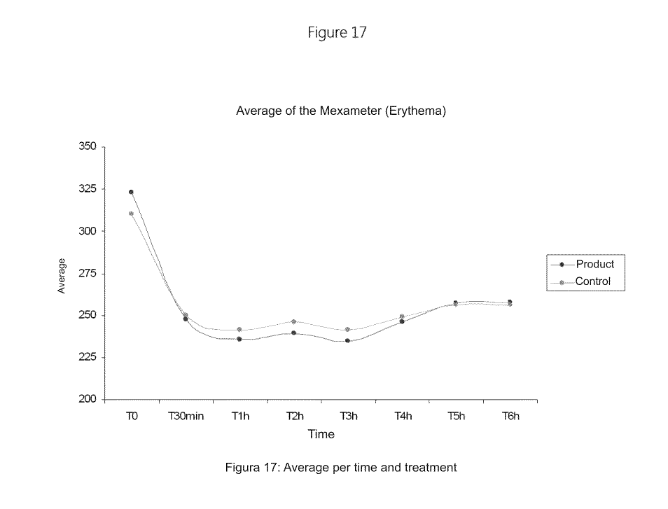

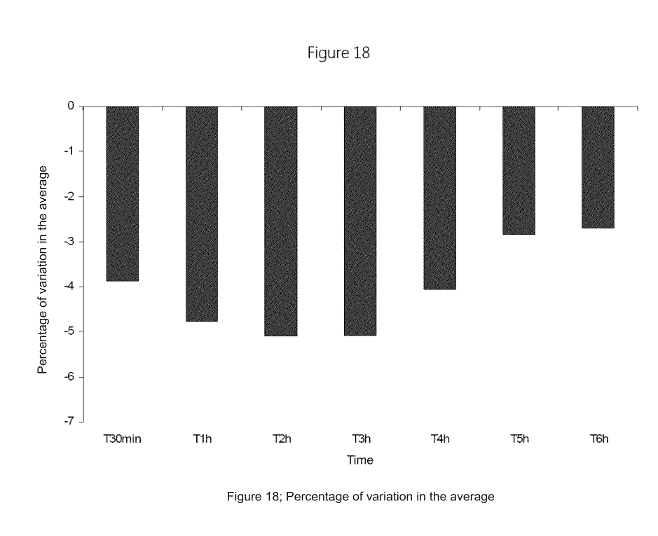

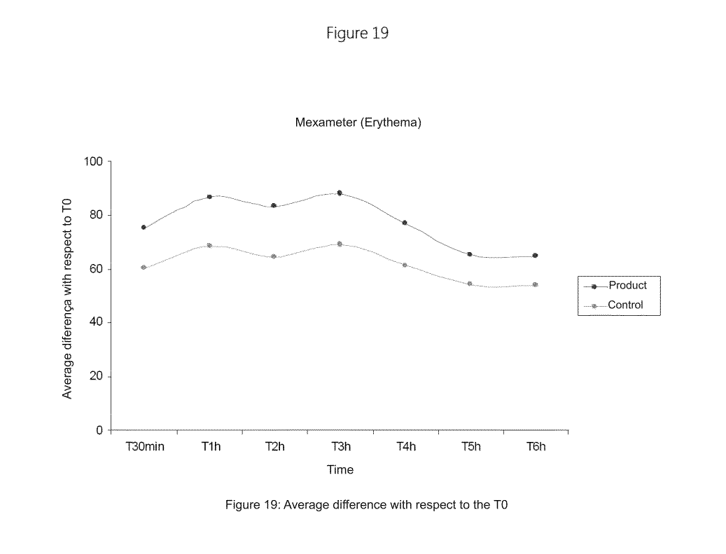

In evaluating the reduction of the erythema by colorimetry, one measured the values of the coordinate *a for each volunteer at the site intended for the product and at the control site. FIG. 15 shows the average value of the Erythema Intensity (+a*).

In order to evaluate the homogeneity of the erythema formed by the mechanical insult, the values of a* measured at each site in the basal time (t0) were statistically compared by using the t-Student test, in pairs, with trust interval of 95%. The results achieved are summarized in table 30.

TABLE 30
Comparison of the values of the erythema occurrences
induced after mechanical insult. Value of P

| | |
|---|---|
| a*t0 product vs. a* t0 control | 0.6682 (non-significant) |

According to the results obtained, there was no statistically significant difference between the sites (P>0.05), indicating that there was homogeneity in the values of a* after the mechanical insult. This indicated that the study was conducted with homogeneous basal erythema values for the sites under evaluation.

In order to evaluate whether there were significant alterations in the erythema along the study, the erythema measurements obtained after 0.5, 1, 2, 3, 4, 5, and 6 hours from application of the product or non-application were compared with the basal measurements (t0) carried out right after the mechanical insult (prior to application of the product), by using the single-factor variance analysis, with post-test of Dunnett multiple comparison, considering α=0.0.05. The results of the statistical analysis are summarized in Table 31.

TABLE 31
Summarized data of the statistical analysis. Comparison of the values
of a* obtained after the mechanical insult (t0) vs. ti (wherein i =
0.5, 1, 2, 3, 4, 5 e 6 h from application), Values of P.

| Comparison group | Control | NT1123-12-A |
|---|---|---|
| a*ₜ₀ vs. a*_{t0.5} | P < 0.05 (significant) | P < 0.05 (significant) |
| a*ₜ₀ vs. a*ₜ₁ | P < 0.05 (significant) | P < 0.05 (significant) |
| a*ₜ₀ vs. a*ₜ₂ | P < 0.05 (significant) | P < 0.05 (significant) |
| a*ₜ₀ vs. a*ₜ₃ | P < 0.05 (significant) | P < 0.05 (significant) |
| a*ₜ₀ vs. a*ₜ₄ | P < 0.05 (significant) | P < 0.05 (significant) |
| a*ₜ₀ vs. a*ₜ₅ | P < 0.05 (significant) | P < 0.05 (significant) |
| a*ₜ₀ vs. a*ₜ₆ | P < 0.05 (significant) | P < 0.05 (significant) |

According to the results, it was possible to observe that that was a statistically significant reduction (P<0.05) of the skin erythema after 0.5, 1, 2, 3, 4, 5, and 6 hours from application of the composition according to the invention as compared with the initial erythema (t0), and that in the control site (without application of any products) there was a significant reduction (P<0.05) in the erythema formed by the mechanical insult after 0.5, 1, 2, 3, 45, and 6 hours. Although there has been a reduction of the erythema at both sites along the study, it is possible to observe, in FIG. 16, which shows the percentage of Reduction of Erythema Intensity (% RIE) obtained in each evaluation time, that there was a greater reduction of the erythema at the site intended for the product.

In order to evaluate the Calming Effect (CE/EC), one calculated the ratio between the measurements of a* after 0.5, 1, 2, 3, 4, 5, and 6 hours with respect to the value obtained along the mechanical insult (t0), by using Equation 2.

EC=a*ₜᵢ/a*ₜ₀

Equation 2—Calculation of the Calming Effect (EC). a*ₜᵢ=measurement of the coloration of the erythema in the time i (i=0.5, 1, 2, 3, 4, 5 e 6h) e a*ₜ₀=measurement of the coloration of the basal erythema (right after formation)

The values of the Calming Effect achieved for the site with application of the product, in each time, were statistically compared with the values achieved for the control by using the t-Student test, bimodal, in pairs, with trust interval of 95%. The results of the statistical analysis are summarized in table 32.

TABLE 32
A comparison of the results in terms of EC between the product and the
respective control. Values of P.

| Comparison | | | | | | | |
|---|---|---|---|---|---|---|---|
| group | t0.5 | t1 | t2 | t3 | t4 | t5 | t6 |
| EC_{NT1123-12-A} | 0.0358 | 0.0280 | 0.5951 | 0.4005 | 0.5370 | 0.7799 | 0.6547 |
| vs. EC_{Controle} | (significant) | (significant) | (non- | (non- | (non- | (non- | (non- |
| | | | sitnificant) | significant) | significant) | significant) | significant) |

The reduction of the skin erythema provided by the composition according to the invention shows a statistically significant difference (P<0.05) after 30 minutes and 1 hour from application as compared with the respective control (site without application of any products). This indicated that the product was effective in reducing the skin erythema induced by mechanical insult and evaluated as a function of the skin redness until 1 hour from application.

Test 6—Instrumental Test of the Effect of the Composition of the Invention (Called 12-33171-07) on the Erythema Caused by the Tape-Stripping Technique

### 6.1—Objective

Evaluating the potential of calming action of a topical product referring to a composition according to the invention through instrumental measurements of colorimetry.

6.2—Methodology. Materials and Equipment

Two symmetrical 10 cm² areas having randomized distribution in the front region of the forearms of the patients (a total of 19 participants) were demarcated. One of the areas was used for application of the product and the other was kept as control (untreated area). Colorimetric measurements were made with the equipment Mexameter MX18-Courage+Khazaka prior to tape-stripping removal (Fita Hipoalergênica Transproe^{MR}) and application of the product (Tb) after 30 tape-stripping removals at each site (T0) and after 30 minutes, 1, 2, 3, 4, 5 and 6 hours from application of the product.

### 6.3—Measurement of Erythema-Mexametry

The measurements were carried out by using the equipment Mexameter MX 18, Courage+Khazaka electronic GmbH through a measurement probe. The readings were made by applying the probe to the test areas with the pressure permitted by the spring (0.5 N).

The measurement area was 5 mm in diameter. Three measurements were carried out in each area. The measurement consisted in measuring the light absorbed and reflected at the wavelengths for green and red for hemoglobin and wavelengths for green and near infrared for melanin.

The operator positioned the probe vertically, forming a 90-degree angle with the skin and cleaned the probe with the aid of a very soft piece of paper prior to the first reading and between the readings of one area an another, even if it were the control area or the initial measurement of each area.

The reading indicated the degree of erythema of the skin. The scale of the equipment is arbitrary, the reading values indicating greater redness of the skin (erythema).

### 6.4—Steps of Research

### 6.4.1—First Step

The participants remained at rest in an air-conditioned room with temperature of 20±2° C. and relative humidity of 50±5% for at least 30 minutes prior to each reading;
The participants were told not to smoke; not to come out of the test room without prior authorization of the expert; not to come into contact with the area being tested at any place; not to contact the area being tested in contact with the clothes between the first air-conditioning and the end of the measurements; not to make abrupt movements with any part of the body; and not to allow the test area to get wet
The participants were evaluated by the dermatologist to confirm the research inclusion criteria;
Two symmetric 10 cm² areas of the region before the forearms having randomized distribution were demarcated. One area was used for application of the product and the other area was kept as negative control (untreated area); and
One determined the coloration of the areas through arithmetic mean of three measurements (Tb).

### 6.4.2—Second Step

30 tape-stripping removals were carried out in the two demarcated areas. The adhesive tapes (TransporeMR) were replaced at each removal; and
One determined the coloration of the demarcated areas through the arithmetic mean of three measurements (T0).

### 6.4.3—Third Step

The tested product was applied in a randomized manner, in the amount of 0.02 g in the demarcated region of each participant. The product was spread over the skin with the aid of a latex fingerstall, with light and circular movements until the whole application area was entirely covered and homogeneous. The latex fingerstall was changed in each area;
The measurements were carried out in the following times:
T30min—thirty minutes after application of the product
T1h—one hour after application of the product
T2h—two hours after application of the product
T3h—three hours after application of the product;
T4h—four hours after application of the product;
T5h—five hours after application of the product;
T6h—six hours after application of the product.
The participants were evaluated by the dermatologist at the end of the measurements T6h; and
After the medical evaluation the participants were released.

### 6.5—Statistical Analysis

The software used in the analyses was MINITAB 14 AND XLSTAT 2012.

As said before, the number of participants in the research was 19

The trust level considered in the comparative analysis was of 95%.

### 6.6—Results

### 6.6.1—Erythema Measurement-Mexametry

TABLE 33
Measurements descriptive statistics and results of the comparison

| | 12-33171-07 | | Control | | 12-33171-07 | Control |
|---|---|---|---|---|---|---|
| Subject | Tb | T0 | Tb | T0 | Δ (T0 − Tb) | Δ (T0 − Tb) |
| 002 | 222 | 325 | 224 | 291 | 103 | 67 |
| 004 | 262 | 293 | 264 | 322 | 31 | 58 |
| 005 | 173 | 291 | 198 | 256 | 118 | 58 |
| 006 | 255 | 346 | 267 | 323 | 91 | 56 |
| 007 | 147 | 346 | 174 | 213 | 199 | 39 |
| 008 | 238 | 369 | 246 | 338 | 131 | 92 |
| 009 | 209 | 258 | 209 | 334 | 49 | 125 |
| 010 | 157 | 266 | 192 | 172 | 109 | −20 |
| 012 | 187 | 269 | 162 | 285 | 82 | 123 |
| 013 | 328 | 383 | 340 | 364 | 55 | 24 |
| 014 | 163 | 264 | 117 | 258 | 101 | 141 |
| 015 | 137 | 304 | 163 | 236 | 167 | 73 |
| 016 | 155 | 293 | 135 | 351 | 138 | 216 |
| 017 | 205 | 363 | 193 | 397 | 158 | 204 |
| 018 | 279 | 413 | 271 | 368 | 134 | 97 |
| 019 | 244 | 380 | 227 | 374 | 136 | 147 |
| 020 | 208 | 308 | 243 | 319 | 100 | 76 |
| 021 | 212 | 287 | 229 | 313 | 75 | 84 |
| 022 | 293 | 374 | 330 | 382 | 81 | 52 |
| Average | 214.4 | 322.7 | 220.2 | 310.3 | 108.3 | 90.1 |
| Medium | 209.0 | 308.0 | 224.0 | 322.0 | 103.0 | 76.0 |
| Minimum | 137.0 | 258.0 | 117.0 | 172.0 | 31.0 | −20.0 |
| Maximum | 328.0 | 413.0 | 340.0 | 397.0 | 199.0 | 216.0 |
| Standard erros | 12.2 | 10.9 | 13.6 | 14.0 | 9.7 | 13.4 |
| IC of 95% | [189.9; | [300.9; | [193; 247.4] | [282.3; | [88.9; 127.8] | [63.2; 117] |
| | 238.9] | 344.6] | | 338.3] | | |

| | | |
|---|---|---|
| Δ (%) with respect to the T0 | 50.5 | 40.9 |
| % of volunteers with irritating effect | 100.0 | 94.7 |
| P-Value | <0.001*** | <0.001*** |

***significant at level 0.1%;
**significant at level 1%;
*significant at level 5% (t-Student test).

The time T0 was higher on the average than the time Tb for product and control

TABLE 34
Measurements and descriptive statistics of the product

| | | | | | | | | | Δ (T30- |
|---|---|---|---|---|---|---|---|---|---|
| | | T30- | | | | | | | min − |
| Subject | T0 | min | T1 h | T2 h | T3 h | T4 h | T5 h | T6 h | T0) |
| 002 | 325 | 214 | 210 | 204 | 224 | 248 | 248 | 227 | −111 |
| 004 | 293 | 323 | 320 | 284 | 309 | 296 | 280 | 311 | 30 |
| 005 | 291 | 198 | 191 | 194 | 191 | 207 | 203 | 213 | −93 |
| 006 | 346 | 289 | 232 | 259 | 243 | 262 | 240 | 245 | −57 |
| 007 | 346 | 209 | 202 | 220 | 269 | 208 | 251 | 277 | −137 |
| 008 | 369 | 278 | 269 | 267 | 266 | 250 | 264 | 242 | −91 |
| 009 | 258 | 235 | 192 | 187 | 190 | 186 | 228 | 182 | −23 |
| 010 | 266 | 193 | 190 | 229 | 225 | 227 | 231 | 233 | −73 |
| 012 | 269 | 245 | 212 | 211 | 209 | 226 | 302 | 265 | −24 |
| 013 | 383 | 276 | 319 | 325 | 320 | 305 | 336 | 350 | −107 |
| 014 | 264 | 149 | 157 | 176 | 178 | 185 | 195 | 195 | −115 |
| 015 | 304 | 200 | 214 | 191 | 194 | 204 | 214 | 244 | −104 |
| 016 | 293 | 229 | 192 | 188 | 183 | 207 | 249 | 265 | −64 |
| 017 | 363 | 225 | 191 | 255 | 206 | 248 | 260 | 249 | −138 |
| 018 | 413 | 345 | 311 | 359 | 325 | 336 | 366 | 347 | −68 |
| 019 | 380 | 335 | 312 | 267 | 255 | 316 | 287 | 308 | −45 |
| 020 | 308 | 230 | 241 | 226 | 166 | 247 | 254 | 231 | −78 |
| 021 | 287 | 191 | 194 | 196 | 176 | 197 | 190 | 225 | −96 |
| 022 | 374 | 340 | 335 | 310 | 328 | 316 | 288 | 290 | −34 |
| Average | 322.7 | 247.6 | 236.0 | 239.4 | 234.6 | 245.8 | 257.2 | 257.8 | −75.2 |
| Medium | 308.0 | 230.0 | 212.0 | 226.0 | 224.0 | 247.0 | 251.0 | 245.0 | −78.0 |
| Minimum | 258.0 | 149.0 | 157.0 | 176.0 | 166.0 | 185.0 | 190.0 | 182.0 | −138.0 |
| Maximum | 413.0 | 345.0 | 335.0 | 359.0 | 328.0 | 336.0 | 366.0 | 350.0 | 30.0 |
| Standard | 10.9 | 13.2 | 12.9 | 12.0 | 12.5 | 10.9 | 10.5 | 10.7 | 9.8 |
| error | | | | | | | | | |
| IC of 95% | [300.9; | [221.2; | [210.1; | [215.4; | [209.5; | [224; | [236.2; | [236.5; | [−94.8; |
| | 344.6] | 273.9] | 261.9] | 263.4] | 259.6] | 267.7] | 278.2] | 279.2] | −55.5] |

| | |
|---|---|
| Δ (%) with respect to T0 | −23.3 |

| | Δ | Δ | Δ | Δ | Δ | Δ |
|---|---|---|---|---|---|---|
| | (T1 h − | (T2 h − | (T3 h − | (T4 h − | (T5 h − | (T6 h − |
| Subject | T0) | T0) | T0) | T0) | T0) | T0) |
| 002 | −115 | −121 | −101 | −77 | −77 | −98 |
| 004 | 27 | −9 | 16 | 3 | −13 | 18 |
| 005 | −100 | −97 | −100 | −84 | −88 | −78 |
| 006 | −114 | −87 | −103 | −84 | −106 | −101 |
| 007 | −144 | −126 | −77 | −138 | −95 | −69 |
| 008 | −100 | −102 | −103 | −119 | −105 | −127 |
| 009 | −66 | −71 | −68 | −72 | −30 | −76 |
| 010 | −76 | −37 | −41 | −39 | −35 | −33 |
| 012 | −57 | −58 | −60 | −43 | 33 | −4 |
| 013 | −64 | −58 | −63 | −78 | −47 | −33 |
| 014 | −107 | −88 | −86 | −79 | −69 | −69 |
| 015 | −90 | −113 | −110 | −100 | −90 | −60 |
| 016 | −101 | −105 | −110 | −86 | −44 | −28 |
| 017 | −172 | −108 | −157 | −115 | −103 | −114 |
| 018 | −102 | −54 | −88 | −77 | −47 | −66 |
| 019 | −68 | −113 | −125 | −64 | −93 | −72 |
| 020 | −67 | −82 | −142 | −61 | −54 | −77 |
| 021 | −93 | −91 | −111 | −90 | −97 | −62 |
| 022 | −39 | −64 | −46 | −58 | −86 | −84 |
| Average | −86.7 | −83.4 | −88.2 | −76.9 | −65.6 | −64.9 |
| Medium | −93.0 | −88.0 | −100.0 | −78.0 | −77.0 | −69.0 |
| Minimum | −172.0 | −126.0 | −157.0 | −138.0 | −106.0 | −127.0 |
| Maximum | 27.0 | −9.0 | 16.0 | 3.0 | 33.0 | 18.0 |
| Standard | 9.6 | 7.1 | 9.1 | 7.2 | 8.5 | 8.3 |
| error | | | | | | |
| IC of 95% | [−105.9; | [−97.5; | [−106.3; | [−91.3; −62.5] | [−82.6; | [−81.5; −48.3] |
| | −67.6] | −69.2] | −70.1] | | −48.5] | |
| Δ (%) with respect | −26.9 | −25.8 | −27.3 | −23.8 | −20.3 | −20.1 |
| to T0 | | | | | | |

TABLE 35
Measurements and descriptive statistics of the control

| | | | | | | | | | Δ (T30- |
|---|---|---|---|---|---|---|---|---|---|
| | | T30- | | | | | | | min − |
| Subject | T0 | min | T1 h | T2 h | T3 h | T4 h | T5 h | T6 h | T0) |
| 002 | 291 | 194 | 214 | 201 | 216 | 245 | 245 | 241 | −97 |
| 004 | 322 | 327 | 326 | 283 | 301 | 318 | 314 | 297 | 5 |
| 005 | 256 | 193 | 178 | 189 | 189 | 189 | 210 | 196 | −63 |
| 006 | 323 | 282 | 242 | 250 | 258 | 248 | 250 | 242 | −41 |
| 007 | 213 | 175 | 181 | 176 | 217 | 198 | 226 | 221 | −38 |
| 008 | 338 | 304 | 297 | 287 | 288 | 267 | 270 | 280 | −34 |
| 009 | 334 | 259 | 231 | 213 | 202 | 176 | 228 | 200 | −75 |
| 010 | 172 | 202 | 166 | 206 | 180 | 200 | 163 | 184 | 30 |
| 012 | 285 | 234 | 204 | 232 | 209 | 230 | 271 | 262 | −51 |
| 013 | 364 | 279 | 299 | 329 | 365 | 329 | 349 | 352 | −85 |
| 014 | 258 | 195 | 205 | 195 | 216 | 185 | 191 | 219 | −63 |
| 015 | 236 | 160 | 151 | 147 | 156 | 212 | 189 | 194 | −76 |
| 016 | 351 | 273 | 292 | 344 | 278 | 257 | 276 | 290 | −78 |
| 017 | 397 | 238 | 218 | 251 | 209 | 257 | 251 | 260 | −159 |
| 018 | 368 | 327 | 267 | 305 | 289 | 272 | 322 | 327 | −41 |
| 019 | 374 | 298 | 290 | 264 | 235 | 329 | 320 | 311 | −76 |
| 020 | 319 | 244 | 274 | 237 | 215 | 259 | 254 | 258 | −75 |
| 021 | 313 | 235 | 235 | 228 | 233 | 248 | 246 | 238 | −78 |
| 022 | 382 | 333 | 322 | 336 | 328 | 312 | 291 | 297 | −49 |
| Average | 310.3 | 250.1 | 241.7 | 245.9 | 241.3 | 249.0 | 256.1 | 256.3 | −60.2 |
| Medium | 322.0 | 244.0 | 235.0 | 237.0 | 217.0 | 248.0 | 251.0 | 258.0 | −63.0 |
| Minimum | 172.0 | 160.0 | 151.0 | 147.0 | 156.0 | 176.0 | 163.0 | 184.0 | −159.0 |
| Maximum | 397.0 | 333.0 | 326.0 | 344.0 | 365.0 | 329.0 | 349.0 | 352.0 | 30.0 |
| Standard | 14.0 | 12.4 | 12.4 | 13.0 | 12.4 | 11.1 | 11.3 | 11.0 | 9.0 |
| error | | | | | | | | | |
| IC of | [282.3; | [225.3; | [216.9; | [220; | [216.5; | [226.7; | [233.4; | [234.2; | [−78.3; |
| 95% | 338.3] | 274.9] | 266.4] | 271.8] | 266.1] | 271.3] | 278.8] | 278.3] | −42.2] |

| | |
|---|---|
| Δ (%) em relação ao T0 | −19.4 |

| | Δ | Δ | Δ | Δ | Δ | Δ |
|---|---|---|---|---|---|---|
| | (T1 h − | (T2 h − | (T3 h − | (T4 h − | (T5 h − | (T6 h − |
| Subject | T0) | T0) | T0) | T0) | T0) | T0) |
| 002 | −77 | −90 | −75 | −46 | −46 | −50 |
| 004 | 4 | −39 | −21 | −4 | −8 | −25 |
| 005 | −78 | −67 | −67 | −67 | −46 | −60 |
| 006 | −81 | −73 | −65 | −75 | −73 | −81 |
| 007 | −32 | −37 | 4 | −15 | 13 | 8 |
| 008 | −41 | −51 | −50 | −71 | −68 | −58 |
| 009 | −103 | −121 | −132 | −158 | −106 | −134 |
| 010 | −6 | 34 | 8 | 28 | −9 | 12 |
| 012 | −81 | −53 | −76 | −55 | −14 | −23 |
| 013 | −65 | −35 | 1 | −35 | −15 | −12 |
| 014 | −53 | −63 | −42 | −73 | −67 | −39 |
| 015 | −85 | −89 | −80 | −24 | −47 | −42 |
| 016 | −59 | −7 | −73 | −94 | −75 | −61 |
| 017 | −179 | −146 | −188 | −140 | −146 | −137 |
| 018 | −101 | −63 | −79 | −96 | −46 | −41 |
| 019 | −84 | −110 | −139 | −45 | −54 | −63 |
| 020 | −45 | −82 | −104 | −60 | −65 | −61 |
| 021 | −78 | −85 | −80 | −65 | −67 | −75 |
| 022 | −60 | −46 | −54 | −70 | −91 | −85 |
| Average | −68.6 | −64.4 | −69.1 | −61.3 | −54.2 | −54.1 |
| Medium | −77.0 | −63.0 | −73.0 | −65.0 | −54.0 | −58.0 |
| Minimum | −179.0 | −146.0 | −188.0 | −158.0 | −146.0 | −137.0 |
| Maximum | 4.0 | 34.0 | 8.0 | 28.0 | 13.0 | 12.0 |
| Standard | 9.0 | 9.4 | 11.4 | 10.0 | 8.7 | 9.0 |
| error | | | | | | |
| IC of | [−86.6; | [−83.1; | [−91.9; −46.2] | [−81.4; −41.3] | [−71.6; | [−72.1; −36] |
| 95% | −50.6] | −45.7] | | | −36.8] | |
| Δ (%) em relação | −22.1 | −20.7 | −22.3 | −19.8 | −17.5 | −17.4 |
| ao T0 | | | | | | |

TABLE 36
Average and standard error for each treatment per
time and for the difference between treatments

| | Product | | Control | | Product − Control | |
|---|---|---|---|---|---|---|
| Time | Average | E.P. | Average | E.P. | Average | E.P. |
| T0 | 322.7 | 10.9 | 310.3 | 14.0 | 12.4 | 11.7 |
| T30 min | 247.6 | 13.2 | 250.1 | 12.4 | −2.5 | 6.1 |
| T1 h | 236.0 | 12.9 | 241.7 | 12.4 | −5.7 | 8.7 |
| T2 h | 239.4 | 12.0 | 245.9 | 13.0 | −6.6 | 10.1 |
| T3 h | 234.6 | 12.5 | 241.3 | 12.4 | −6.7 | 8.7 |
| T4 h | 245.8 | 10.9 | 249.0 | 11.1 | −3.2 | 6.0 |
| T5 h | 257.2 | 10.5 | 256.1 | 11.3 | 1.1 | 6.7 |
| T6 h | 257.8 | 10.7 | 256.3 | 11.0 | 1.6 | 6.2 |

TABLE 37
Porcentage of variation in the average with respect to the T0
and percentage of volunteers with positive and negative effect

| | Produto − Controle | | |
|---|---|---|---|
| | % of | | |
| | variation with | % of subjects with | % of subjects with |
| Time | respect to T0 | positive effect | negative effect |
| T30 min | −3.9 | 63.2 | 36.8 |
| T1 h | −4.8 | 63.2 | 36.8 |
| T2 h | −5.1 | 73.7 | 21.1 |
| T3 h | −5.1 | 68.4 | 21.1 |
| T4 h | −4.1 | 63.2 | 36.8 |
| T5 h | −2.9 | 68.4 | 31.6 |
| T6 h | −2.7 | 63.2 | 36.8 |

No significant difference was found between the treatments in the times T30min, T1h, T4h, T5h and T6h (P-values=0.138; 0.054; 0.147; 0.250; 0.207, respectively). The product was inferior to the control in the times T2h and T3h (p-values =0.048; 00.41, respectively).

### Wherein:

Cm2: square centimeters;
G: grams;
H: hours;
No.: number;
° C.; degrees Celsius;
Tx: Time after x hours from application of the product.

### 6.7—Conclusion

After a statistical analysis of the results, one can conclude that the composition of the invention promoted the reduction of the erythema in the times T2h and T3h as compared with the control.

1. An antiperspirant deodorant cosmetic composition with dermo-calming action characterized by comprising 2-methyl-5-cyclohexylpentanol in an amount ranging from 0.1 percent to 1 percent by weight based on a total weight of the composition, aluminum hydrochloride present as a 50 percent solution in an amount of 5.0 percent to 40 percent by weight based on the total weight of the composition, and cosmetically acceptable adjuvants.

2. The composition according to claim 1, characterized by further containing a conditioning agent and a consistency agent.

3. The composition according to claim 2, characterized in that the conditioning agent is pantenol and the consistency agent is hydroxypropylic starch phosphate.

4. The composition according to claim 1, characterized in that the cosmetically acceptable adjuvants are selected from the group consisting of emulsifying agent, preserving agent, antioxidant agent, sequestering agent, chelating agent, oil, water and fragrance.

5. The composition according to claim 4, characterized in that said sequestering agent is disodium EDTA.

6. The composition according to claim 4, characterized in that said preserving agent is dimethyldimethylhydantoin (DMDM hydantoin).

7. The composition according to claim 4, characterized in that said antioxidant agent is butylated hydroxidetoluene (BHT).

8. The composition according to claim 4, characterized in that said oil is olus oil.

9. The composition according to claim 4, characterized in that said oil is hydrogenated palm oil.

10. The composition according to claim 1, characterized by further comprising silica dimethyl sililate and PPG-15 stearyl ether.

11. The composition according to claim 4, characterized in that said emulsifying agent is selected from the group consisting of steareth-2 and steareth-21.

12. The composition according to claim 1, characterized by further comprising dicapryl carbonate, cyclopentasyloxane, dimethiconol, magnesium silicate, ceresin wax.

13. The composition according to claim 4, characterized in that said emulsifying agent is selected from the group consisting of cetostearyl alcohol and ceteareth-20.

14. The composition according to claim 1, characterized in that it is in the form of roll-on deodorant.

15. The composition according to claim 1, characterized in that it is in the form of a cream deodorant.

16. The composition according to claim 14, after depilation.
